(19) United States
(12) Patent Application Publication
(10) Pub. No.: US 2023/0303646 A1
(43) Pub. Date: Sep. 28, 2023

(54) ANALOGS OF HUMAN FIBROBLAST GROWTH FACTORS

(71) Applicant: ENERGESIS PHARMACEUTICALS, INC., CAMBRIDGE, MA (US)

(72) Inventors: OLIVIER BOSS, BOSTON, MA (US); BRIAN FREEMAN, CAMBRIDGE, MA (US)

(21) Appl. No.: 18/021,561

(22) PCT Filed: Aug. 19, 2021
(86) PCT No.: PCT/US2021/046602

§ 371 Date: Feb. 16, 2023

Related U.S. Application Data
(60) Provisional application No. 63/067,675, filed on Aug. 19, 2020.

(51) Int. Cl. C07K 14/50; A61P 3/00

(52) U.S. Cl. CPC C07K 14/50; A61P 3/00; C07K 2319/30

(57) ABSTRACT

This invention relates to methods for identifying novel compositions recruiting brown adipocytes in vitro and in vivo from brown adipocyte progenitor cells as well as such novel compositions. In some embodiments, the novel composition is a human protein or peptide. In other embodiments the novel composition is an Fc fusion of a human protein or peptide. In still other embodiments, the novel composition is an Fc fusion of human FGF-7.

### TECHNICAL FIELD

The present invention relates to methods and compositions to enhance brown adipocytes, and/or brown adipocyte mass, in conditions such as type 2 diabetes, obesity, insulin-resistance, and dyslipidemia. Specifically, the present invention identifies methods for creating analogs of a human protein that recruits brown adipocytes or increases the differentiation of brown adipose tissue (BAT) progenitor cells into brown adipocytes, which are longer-lasting and safer than the parent protein. Further, the present invention identifies compounds comprising longer-acting, safer analogs of a human protein that recruits brown adipocytes or increases the differentiation of brown adipose tissue (BAT) progenitor cells into brown adipocytes. The invention is useful for the study, prevention, and treatment of various metabolic disorders such as obesity, type 2 diabetes, insulin-resistance and dyslipidemia. Still further, the present invention provides methods for the identification of additional therapeutic compounds for the prevention and treatment of metabolic disorders such as type 2 diabetes, obesity, insulin-resistance, and dyslipidemia.

### BACKGROUND

Applicants propose herein compositions and methods to identify such compositions which are analogs of a compound that has been shown to recruit new brown adipocytes from human progenitor cells and is highly efficacious in an animal model of obesity and insulin resistance (U.S. patent application Ser. No. 15/120,850). Proposed are methods to identify new and improved compounds based on characteristics including but not limited to their pharmacokinetics (PK), manufacturability, and in vivo efficacy, as well as compositions with one or more of these attributes. These methods can be used to discover improved compounds for targeting brown fat useful for the treatment of conditions such as obesity, overweight, and insulin resistance in patients and animals.

The epidemic of obesity is closely associated with increases in the prevalence of diabetes, hypertension, coronary heart disease, cancer and other disorders. The role of white adipose tissue is to store lipids, and it is associated with obesity and other weight disorders. The role of brown adipose tissue (“BAT”) is effectively the opposite of that of white adipose tissue. It is specialized in lipid combustion and the dissipation of energy as heat. Indeed, the brown adipocyte contains numerous mitochondria (in which cellular combustion occurs) and uniquely expresses uncoupling protein-1 (“UCP1”). UCP1 acts as an uncoupler of oxidative phosphorylation, resulting in dissipation of energy as heat. It also acts as a unique marker for the presence or absence of BAT. The sympathetic nervous system stimulates mitochondriogenesis and UCP1 expression and activity. BAT-associated thermogenesis in rodents is increased upon exposure to low temperature (e.g., to prevent hypothermia), or as a result of overeating, in order to burn excess absorbed calories and prevent weight gain. BAT, by modifying susceptibility to weight gain and by consuming large amounts of glucose, also improves insulin sensitivity. It therefore plays an important role in the maintenance of body temperature, energy balance and glucose metabolism.

Experiments with transgenic animals support the potential anti-obesity properties of BAT. For example, the genetic ablation of BAT has been reported to cause obesity, while a genetically-mediated increase in the amount and/or function of BAT (and/or UCP1 expression) reportedly promotes a lean and healthy phenotype. Specifically, mice with a higher amount of BAT gain less weight and are more insulin-sensitive than control mice. Recently, ectopic BAT depots were evidenced in the mouse muscle, which have been shown to provide a genetic mechanism of protection from weight gain and metabolic syndrome.

Although UCP1 is reported to play a role in the control of energy balance in rodents and UCP1-expressing BAT is present in human neonates, it has long been thought that there was no physiologically relevant UCP1 expression in adult humans. Indeed, UCP1-expressing BAT was thought to disappear early in life, and adult humans were thought to be devoid of BAT. Recently however, numerous studies have demonstrated that UCP1 is expressed and BAT is indeed maintained in most adult humans, albeit at considerably lower levels than in neonates and children.

Hence, a need exists to carefully identify ways to provide more BAT in the adult body and/or stimulate UCP1 expression, for the study, prevention and treatment of various metabolic diseases such as obesity, type 2 diabetes, insulin-resistance, dyslipidemia and type 1 diabetes.

Applicants previously identified compounds, including proteins, capable of differentiating brown adipocyte progenitors into brown adipocytes. In one aspect, Applicants identified a human protein capable of differentiating brown adipocyte progenitors into brown adipocytes. In another aspect, Applicants identified a human protein capable of differentiating brown adipocyte progenitors isolated from human skeletal muscle into brown adipocytes. In yet another aspect, Applicants identified a human protein capable of differentiating brown adipocyte progenitors, which are perivascular in location and isolated from human skeletal muscle into brown adipocytes. The previous disclosure provided for the use of such protein in the manufacture of a medicament and for the use of such medicament in modulating a metabolic response in a subject or for use in preventing or treating a metabolic disorder in a subject. Applicants obtained this protein, Fibroblast Growth Factor 7 (FGF-7, commercially available and tested in a stabilized form as Kepivance (palifermin) and referred to by company code EGS0501, SEQ ID NO: 1), to evaluate its potential in a predictive animal model of obesity and insulin resistance, the DIO (Diet Induced Obese) mouse. EGS0501 is a truncated form of the full length FGF-7 human protein (SEQ ID NO: 13; Genbank), which is fully capable of differentiating brown adipocyte progenitors isolated from human skeletal muscle into brown adipocytes. This form of the protein has a longer plasma half-life than full length FGF-7 protein but retains the full activity of the parent molecule. Applicants demonstrated that EGS0501 is very efficacious, producing highly significant improvements in body weight, body composition and glucose handling during a 28 day study, without affecting lean body mass.

Applicants previously identified additional Fibroblast Growth Factor family members, specifically Fibroblast Growth Factor 10 and Fibroblast Growth Factor 13, which also have the ability to recruit human brown adipocytes. The invention described herein may be used for any of these Fibroblast Growth Factor proteins.

### SUMMARY

The present invention provides compositions, and methods for discovering, improved analogs of human FGF-7. EGS0501 has an average half-life (T_{1/2}) of only 4.5 hours in humans. Compounds for use in obesity and diabetes that have a longer T_{1/2}, such as liraglutide (13 hours), Lantus, and other insulin glargine products (18-26 hours) are highly useful and more convenient for administration to patients and/or companion animals.

Several techniques for prolonging the half-life of an underlying protein have been developed and can be used for discovering improved analogs of human FGF-7 and the other Fibroblast Growth Factor proteins previously described. These include, but are not limited to, PEGylation, fusion to immunoglobulin Fc domains, fusion to Human Serum Albumin (HSA), fusion to human transferrin, genetic fusion of non-exact repeat peptide sequence (XTENylation, also known as rPEG), fusion to CTP peptide from human chorionic gonadotrophin β-subunit (CTP fusion), fusion to elastin-like peptide (ELPylation), fusion with artificial GLK (gelatin-like protein; GLK fusion), fusion to HAP homo-amino acid polymer (HAPylation), and fusion to proline-alanine-serine polymer (PASylation).

Fc fusion proteins are chimeric proteins containing a human IgG Fc sequence linked to a protein of interest. These molecules generally have much longer T_{1/2} than the underlying protein (up to 2+weeks). There are now more than ten such proteins approved in the US, the largest selling of which is etanercept (Enbrel), approved in 1998. Fc fusion has led to many successful new products.

The properties of fusion proteins can vary widely versus the underlying protein depending on many factors, including the orientation of the protein of interest, fusion to the N-terminal or C-terminal of the Fc moiety, the nature of the linker sequence, and oligomerization of the fusion protein (a traditional Fc fusion, like a full antibody, has two chains linked by a disulfide bond and contains two copies of the underlying protein). Using an iterative process Applicants designed diverse DNA constructs corresponding to human FGF-7-Fc fusion proteins. These Fc fusion variants were transiently transfected into a mammalian protein expression system, and the properties of the constructs were evaluated. Yield was first measured, as this is a critical parameter in the manufacturability and commercial viability of a protein drug. Engagement with the target and in vitro activity was then measured in an appropriate in vitro assay. The PK of the various constructs was determined in an appropriate animal model. The efficacy of the constructs was evaluated in an appropriate animal disease model capable of accepting human protein sequences. Other properties may also be measured, for example aggregation. Aggregation of Fc fusion proteins has consequences for decreasing production and has been implicated in immunogenicity. The mechanisms of protein aggregation vary depending on the protein, linker, and the expression system utilized, and it is difficult a priori to predict which hypothetical Fc fusions of a particular protein will exhibit unacceptable aggregation into high molecular weight species. Techniques to study the degree of aggregation of expressed Fc fusions of human FGF-7 can be used, such as size exclusion chromatography. It is not clear prior to performing the necessary experiments whether a given Fc fusion of a parent protein will have sufficient yield, which may be due to aggregation reducing yield, and therefore in any program to develop Fc fusions of a parent protein to create candidate therapeutic compounds a number of constructs must be created and expressed at pilot scale.

Fc fusion candidate molecules with adequate yield, i.e., with good potential for efficient and cost-effective manufacturability, were evaluated for in vitro activity. Applicants employed an existing assay for measuring FGF-7 activity based on the survival/proliferation of 4MBr-5 cells, an epithelial cell line derived from the lung/bronchus of the Rhesus monkey. Compounds with activity equal to or greater than 75% of EGS0501 were considered to have adequate in vitro activity to progress. While it would not be clear a priori that Fc fusion variants with adequate yield and sufficient activity could be identified, such constructs meeting predetermined criteria were identified and were further evaluated.

Candidates were subsequently evaluated in pharmacokinetic (PK) studies in lean C57BL6 mice to determine circulating T_{1/2}. Several constructs that met pre-determined criteria for adequate circulating half-life were identified and advanced for further evaluation.

Those Fc fusions demonstrating adequate PK were subsequently tested for efficacy in vivo. Fusions resulting in loss of at least 10% total body weight were considered adequate for further evaluation.

The addition of a large moiety to a parent protein yields polypeptide variants of the parent with higher molecular weight. Techniques that can accomplish this include, but are not limited to, PEGylation, fusion to immunoglobulin Fc domains, fusion to Human Serum Albumin (HSA), fusion to human transferrin, genetic fusion of non-exact repeat peptide sequence (XTENylation, also known as rPEG), fusion to CTP peptide from human chorionic gonadotrophin β-subunit (CTP fusion), fusion to elastin-like peptide (ELPylation), fusion with artificial GLK (gelatin-like protein; GLK fusion), fusion to HAP homo-amino acid polymer (HAPylation), and fusion to proline-alanine-serine polymer (PASylation). Larger molecular weight molecules in the circulatory system have a lower propensity to exit the vasculature and enter peripheral tissues. The brown adipocyte progenitor cells previously described by Applicants are found in a peri-vascular location. A strategy of increasing a compound's molecular weight would therefore improve the compound's ability to remain within the vasculature where it can recruit new brown adipocytes, but away from parenchymal tissues where it may mediate toxicity, thereby increasing the therapeutic index over that of the parent molecule.

Applicants have discovered a uniquely useful human protein able to recruit new brown adipocytes from brown fat progenitor cells and produce impressive weight loss and anti-diabetic effects in a highly predictive animal model of obesity and insulin resistance. The tested molecule has a relatively short half-life in vivo. Applicants describe herein compositions and methods for creating and developing novel Fc fusion analogs of this protein with a longer half-life, good manufacturability, and adequate in vivo efficacy.

Since brown adipose tissue (BAT) is specialized for energy expenditure, the methods described herein are useful for the treatment of obesity and related disorders, such as diabetes. The methods can also be used to decrease fat stores in subjects including food animals, e.g., to improve the quality of the meat derived therefrom.

Accordingly, in one aspect, the invention features methods of treating a subject, e.g., decreasing fat stores or weight in a subject such as a human. The methods include administering to the subject a compound or combination of compounds, at least one of which is identified from the disclosed compositions or methods. The methods can further include administering a compound or compounds to a companion animal in need of decreasing fat stores or weight. The methods can optionally include identifying a subject in need of decreasing fat stores or weight. In a further aspect, the invention includes methods of enhancing insulin sensitivity in a subject, e.g., a subject that is insulin-resistant. The methods include administering to the subject a compound and can optionally include identifying a subject in need of enhanced insulin sensitivity.

In another aspect, the invention features methods of modulating brown adipose tissue function or development, e.g., promoting BAT adipogenesis, in a subject. The methods include administering to the subject a compound or combination of compounds discovered using the disclosed methods.

In general, the subject is a mammal. In some embodiments, the subject is a human subject, preferably an obese or insulin resistant human subject. In some embodiments, the subject is a non-human mammal, e.g., an experimental animal, a companion animal, or a food animal, e.g., a cow, pig, or sheep that is raised for food.

In some embodiments, the methods include evaluating the subject for one or more of: weight, white adipose tissue stores, brown adipose tissue stores, adipose tissue morphology, insulin levels, insulin metabolism, glucose levels, thermogenic capacity, and cold sensitivity. The evaluation can be performed before, during, and/or after the administration of the compound or compounds. For example, the evaluation can be performed at least 1 day, 2 days, 4, 7, 14, 21, 30 or more days before and/or after the administration.

In some embodiments, the methods include one or more additional rounds of treatment with a compound or compounds, e.g., to increase brown adipocyte mass, or e.g., to maintain or further reduce obesity in the subject.

In other embodiments, the methods include treatment using intermittent administration with a compound or compounds, e.g., to increase brown adipocyte mass, or e.g., to maintain or further reduce obesity in the subject.

In certain embodiments of novel FGF-7-Fc fusion proteins, the FGF-7 polypeptide may be fused to the Fc fragment via a peptide linker. Alternatively, no peptide linker may be present between the FGF-7 polypeptide and the Fc domain of the FGF-7-Fc fusion protein (e.g., the C-terminal region of the FGF-7 polypeptide is covalently linked (e.g., via a peptide bond) to the N-terminal region of the Fc domain or the N-terminal region of the FGF-7 polypeptide is covalently linked (e.g., via a peptide bond) to the C-terminal region of the Fc domain). Additionally or alternatively, in some embodiments of the FGF-7-Fc fusion protein, the Fc domain comprises a wild-type Fc fragment of human IgG subtype 1, or subtype 2, or subtype 4.

Additionally or alternatively, in any of the above embodiments of the FGF-7-Fc fusion protein, the FGF-7 polypeptide may be located at the N-terminus or C-terminus of the Fc domain.

In one aspect, the present disclosure provides vectors comprising the recombinant nucleic acid sequences disclosed herein, as well as engineered eukaryotic cells that comprise such vectors (e.g., transfected with a recombinant nucleic acid sequence (e.g., mRNA, cDNA, DNA) encoding an FGF-7-Fc fusion protein described herein.

Additionally or alternatively, in some embodiments of the methods disclosed herein, the FGF-7-Fc fusion protein is administered parenterally, intravenously or subcutaneously. In some embodiments, the FGF-7-Fc fusion protein is administered as an injectable depot formulation. In other embodiments, the FGF-7-Fc fusion protein is administered as a bolus infusion or an intravenous push. In certain embodiments, the FGF-7-Fc fusion protein is administered through syringe injection, pump, pen, needle, or indwelling catheter. The FGF-7-Fc fusion protein may be administered as a single dose or in multiple doses. In certain embodiments, the FGF-7-Fc fusion protein is administered daily, twice daily, twice weekly, or at least weekly to the subject.

Also disclosed herein are kits comprising the FGF-7-Fc fusion protein of the present technology, and instructions for use.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

### BRIEF DESCRIPTION OF THE DRAWINGS

FIG. 1 shows the expression yield of various Fc fusion constructs in CHO cells.

FIG. 2 shows the in vitro activity of various constructs (4MBr-5 cell survival assay).

FIG. 3 shows the plasma half-life of various constructs in Cd57B1/6J mice.

FIG. 4 shows body weight with 28 days of dosing of various constructs (% of baseline weight).

FIG. 5 shows body weight with 28 days of dosing of various constructs (grams).

FIG. 6 shows the weight of the epididymal adipose depot.

FIG. 7 shows food intake (cumulative).

FIG. 8 shows leptin (plasma, 6h fasted), Baseline (d-9) and Terminal (d29).

FIG. 9 shows glucose [mM] (plasma, 6h fasted), Baseline (d-9) and Terminal (d29).

FIG. 10 shows insulin [ng/ml] (plasma, 6h fasted), Baseline (d-9) and Terminal (d29).

FIG. 11 shows HOMA-IR (plasma, 6h fasted), Baseline (d-9) and Terminal (d29).

FIG. 12 shows body weight, as percentage of baseline.

FIG. 13 shows weight of the epididymal adipose depot.

FIG. 14 shows leptin (plasma, 6h fasted), Baseline (d-9), End of dosing (d29), and Recovery for 26d (d55) and 47d (d76).

FIG. 15 shows glucose [mM] (plasma, 6h fasted), Baseline (d-9), End of dosing (d29) and Recovery for 26d (d55) and 47d (d76).

FIG. 16 show insulin [ng/ml] (plasma, 6h fasted), Baseline (d-9), End of dosing (d29) and Recovery for 26d (d55) and 47d (d76).

FIG. 17 shows HOMA-IR (plasma, 6h fasted), Baseline (d-9), End of dosing (d29) and Recovery for 26d (d55) and 47d (d76).

FIG. 18 shows energy expenditure assessed over 24 h after 25 days of dosing, adjusted for body weight based on respective regression analyses. EGS373 led to a significant increase in metabolic rate (24-h energy expenditure).

FIG. 19 shows energy expenditure assessed after 25 days of dosing over 5 hours post-injection of CL316243 (1 mg/kg IP), adjusted for body weight based on respective regression analyses. EGS373 led to a significant increase in thermogenic capacity, reflecting whole-body brown fat mass.

FIG. 20 shows that EGS373 produced a highly significant reduction in body weight in B6.scid DIO mice over 28 days.

FIG. 21 shows that EGS373 dramatically reduced plasma leptin levels, an index of total body fat.

FIG. 22 shows that EGS373 exhibited a trend toward reduced plasma glucose.

FIG. 23 shows that EGS373 significantly reduced plasma insulin.

FIG. 24 shows that HOMA-IR, a particularly sensitive and robust measure of insulin resistance based on plasma insulin and glucose levels, was reduced by EGS373.

FIG. 25 shows food intake, as measured using CLAMS.

FIG. 26 shows locomotor activity, as measured using CLAMS.

FIG. 27 shows ambulatory activity, as measured using CLAMS.

FIG. 28 shows body weight, as a percentage of baseline.

FIG. 29 shows body fat, in grams (by echoMRI).

FIG. 30 shows cumulative food intake (grams/mouse).

FIG. 31 shows leptin (plasma, 6h fasted), Baseline (d-3) and Terminal (d28).

FIG. 32 shows glucose [mM] (plasma, 6h fasted), Baseline (d-3) and Terminal (d28).

FIG. 33 shows insulin [ng/ml] (plasma, 6h fasted), Baseline (d-3) and Terminal (d28).

FIG. 34 shows HOMA-IR (plasma, 6h fasted), Baseline (d-3) and Terminal (d28).

FIG. 35 shows food intake, cumulative.

FIG. 36 shows kaolin consumption over 3 days following cisplatin injection.

FIG. 37 shows a table that correlates construct designations with SEQ ID NOs.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) and Gene Transfer and Expression in Mammalian Cells.

The present disclosure relates to compositions of FGF7-Fc fusion proteins and their use to treat or prevent metabolic disease, e.g., obesity, type 2 diabetes, etc. As described herein, the FGF7-Fc fusion proteins of the present technology recruit, i.e., create new brown adipocytes from brown adipocyte precursor cells and have a long circulating half-life, this avoiding the drawback of FGF7 itself, which has a circulating half-life of only a few hours. Additionally, the FGF7-Fc fusion proteins of the present technology have a lower volume of distribution, permitting access to the perivacular brown adipocyte precursors and reducing access to peripheral tissues of toxicity. Without wishing to be bound by theory, it is believed that in some embodiments, the fusion proteins described herein reduce the toxicity of FGF7.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, 10%, or 20% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, transdermally, or topically. Administration includes self-administration and the administration by another.

As used herein, the term “analog” refers to a compound or conjugate (e.g., a compound, conjugate as described herein, e.g., insulin) having a chemical structure similar to that of another compound or conjugate, but differing from it in at least one aspect.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the treatment of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the pharmaceutical compositions may be administered to a subject having one or more signs or symptoms of a metabolic disease (e.g., obesity, e.g., Type 2 diabetes). As used herein, a “therapeutically effective amount” of a composition refers to composition levels in which the physiological effects of a disease or condition described herein are ameliorated or eliminated. A therapeutically effective amount can be given in one or more administrations. As used herein, a “prophylactically effective amount” of a composition refers to composition levels that prevent or delay the onset of at least one symptom of a disease or condition described herein. A prophylactically effective amount can be given in one or more administrations.

As used herein, the term “fusion protein”, e.g., “FGF7-Fc fusion” protein refers to a protein comprising more than one domain, e.g., typically from different sources (e.g., different proteins, polypeptides, cells, etc.), that are covalently linked through peptide bonds. In some embodiments, a fusion protein is produced recombinantly. In some embodiments, the domains of a fusion protein are covalently linked by connecting the gene sequences that encode each domain into a single nucleic acid molecule. In some embodiments, an FGF7-Fc fusion protein is a protein, e.g., a single polypeptide, comprising an FGF7 polypeptide (e.g., human FGF7, e.g., palifermin) and an Fc fragment polypeptide, where the FGF7 and Fc fragment polypeptides are joined by peptide bonds to form a single polypeptide.

As used herein, the term “FGF7” encompasses mature FGF7 as well as naturally occurring FGF7 or analogs thereof. In some embodiments, an FGF7 polypeptide can be a full-length FGF7 polypeptide or a fragment thereof In some embodiments, an FGF7 polypeptide comprises one or more fragments or domains from a naturally occurring FGF7 and/or one or more fragments or domains from a non-naturally occurring FGF7.

As used herein, the terms “individual”, “patient”, or “subject” can be an individual organism, a vertebrate, a mammal, or a human. In some embodiments, the individual, patient or subject is a human. Exemplary human subjects include a human patient having a disorder, e.g., a disorder described herein, or a normal subject.

The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein means a pharmaceutically acceptable material, composition or vehicle, such as a liquid or solid filler, diluent, excipient, solvent or encapsulating material, involved in carrying or transporting the FGF7-Fc fusion proteins of the present technology from one organ, or portion of the body, to another organ, or portion of the body. Each carrier must be “acceptable” in the sense of being compatible with the other ingredients of the formulation and not injurious to the patient.

As used herein, the term “sample” means biological sample material derived from living cells of a subject. Biological samples may include tissues, cells, protein or membrane extracts of cells, and biological fluids (e.g., ascites fluid or cerebrospinal fluid (CSF)) isolated from a subject, as well as tissues, cells and fluids (blood, plasma, saliva, urine, serum, etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The terms “systemic administration,” “administered systemically,” “peripheral administration” and “administered peripherally” as used herein mean the administration of the FGF7-Fc fusion protein other than directly into the central nervous system, such that it enters the patient's system and, thus, is subject to metabolism and other like processes, for example, subcutaneous administration.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of a disease or disorder as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Management of Obesity

There are 2 main approaches to addressing obesity: 1) reducing energy intake, through the use of agents such as appetite suppressants, lipase inhibitors, or GLP-1 receptor agonists and devices such as intragastric balloons, and 2) increasing energy expenditure. While many products to reduce energy intake have been marketed, there are no approved drugs that specifically enhance energy expenditure.

Thus, there is a need for treatments and approaches for obesity, weight maintenance, and prophylaxis of diabetes, obesity, and other metabolic diseases based on the enhancement of energy expenditure.

### Fc Domains

The term “Fc region”, “Fc domain”, or “Fc fragment” as used herein refers to a C-terminal region of an immunoglobulin heavy chain, which is capable of binding to a mammalian Fc(gamma) or Fc(Rn) receptor, e.g., human Fc(gamma) or Fc(Rn) receptor. An Fc receptor (FcR) refers to a receptor that binds to an Fc fragment or the Fc region of an antibody. In certain embodiments, the FcR is a native human FcR sequence. In some embodiments, the FcR binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are described in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976 J. Immunol., 117:587, and Kim et al., 1994, J. Immunol., 24:249) and contributes to the prolonged in vivo elimination half-lives of antibodies and Fc-fusion proteins in vivo.

The Fc fragment, region, or domain may be a native sequence Fc region. Although the boundaries of the Fc region of an immunoglobulin heavy chain might vary, the human IgG heavy chain Fc region is usually defined to stretch from an ammo acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

In some embodiments, the Fc fragment comprises or consists of the Fc region (e.g., CR2 domain and CH3 domain) of a mammalian IgG, e.g., human IgG. In certain embodiments, the Fc fragment comprises or consists of the Fc region (e.g., CH2 domain and CH3 domain) of human IgG₁. In some embodiments, the Fc fragment comprises or consists of an amino acid sequence having at least 80% (e.g., at least 80%, 85%, 90%, 95%, 97%, 99%, or more) identity to the Fc region (e.g., CH2 domain and CH3 domain) of human IgG₁.

In some embodiments, the Fc region of a human IgG₁ comprises the following amino acid sequence: SEQ ID NO: 8.

In certain embodiments, the Fc region of a human IgG₁ comprises an additional amino acid at one or both termini. In some embodiments, this additional amino acid comprises a charged side chain (e.g., a positively charged amino acid, e.g., lysine or arginine). In certain embodiments, the Fc region of a human IgG₁ comprises the following amino acid sequence: SEQ ID NO: 9.

In certain embodiments, amino acid substitutions can be made to Fc sequences to reduce antibody dependent cellular cytotoxicity (ADCC) mediated through different types of human Fc gamma receptor interactions. Without being limiting an example of such a substitution is N297A. In certain embodiments the Fc region of a human IgG₁ comprises the following amino acid sequence: SEQ ID NO: 10.

In certain embodiments, the Fc fragment comprises or consists of the Fc region (e.g., CH2 domain and CH3 domain) of human IgG₂. In some embodiments, the Fc fragment comprises or consists of an amino acid sequence having at least 80% (e.g., at least 80%, 85%, 90%, 95%, 97%, 99%, or more) identity to the Fc region (e.g., CH2 domain and CH3 domain) of human IgG2.

In some embodiments, the Fc region of a human IgG₂ comprises the following amino acid sequence: SEQ ID NO: 11.

In certain embodiments, the Fc fragment comprises or consists of the Fc region (e.g., CH2 domain and CH3 domain) of human IgG-4. In some embodiments, the Fc fragment comprises or consists of an amino acid sequence having at least 80% (e.g., at least 80%, 85%, 90%, 95%, 97%, 99%, or more) identity to the Fc region (e.g., CH2 domain and CH3 domain) of human IgG4.

In some embodiments, the Fc region of a human IgG4 comprises the following amino acid sequence: SEQ ID NO: 12.

### Fibroblast Growth Factor-7

FGF-7 (Fibroblast Growth Factor-7), also known as KGF (Keratinocyte Growth Factor), is a potent mitogen for different types of epithelial cells, which regulates migration and differentiation of these cells and protects them from various insults under stress conditions. FGF-7 is produced by mesenchymal cells and exerts its biological effects via binding to its high-affinity receptor, a splice variant of FGF receptor 2 (FGFR2-IIIb), which is expressed by various types of epithelial cells, including epidermal keratinocytes, intestinal epithelial cells, and hepatocytes. This expression pattern of FGF-7 and its receptor suggests that FGF-7 acts predominantly in a paracrine manner.

In the present invention, variants, fragments, mutants, derivatives, or functional analogues possess the same pharmacological activity as the FGF-7 protein.

Preferably the FGF-7 variant or orthologs, derivatives, and fragments thereof has at least one residue replaced by a different amino acid residue.

The FGF-7 variants of the present invention, obtained by technologies known in the art, are mutant proteins, which differ from the amino acid sequence of the wild type FGF-7 by the mutation of one or more single ammo acid. In a very preferred embodiment of the present invention, only one amino acid replacement occurs on the sequence of the native protein. It is, however, encompassed by the subject of the present invention that the native protein can be further optimized by replacement of a plurality, e.g., two or more, of amino acid replacements. The variants can therefore differ from the wild type protein sequence by amino acid replacements on 1-10, preferably 1, 2, 3, 4, 5 and 6 different amino acid target positions.

Moreover, the mutants or variants of the invention exhibit the same pharmacological activity as the wild type FGF-7 protein.

The term “mutation” or “variant” as used in the context of the present invention can be understood as substitution, deletion and/or addition of single amino acid in the target sequence.

Preferably, the mutation of the target sequence in the present invention is a substitution. The substitution can occur with different genetically encoded amino acid or by non-genetically encoded amino acids. Examples for non-genetically encoded ammo acids are homocystein, hydroxyproline, omithin, hydroxylysine, citrulline, carnitine, etc.

In some embodiments, the FGF-7 of the present disclosure is a monomer. In some embodiments, the FGF-7 is a non-covalent inultimer (e.g., a dimer, tetramer, hexamer, or higher order multimer. a trimer of dimers). In some embodiments, the FGF-7 may be a monomer or a non-covalent multimer (e.g., a dimer, tetramer, hexamer, or higher order multimer, e.g., a trimer of dimers).

In some embodiments, the FGF-7 described herein is a single chain FGF-7. All salt forms and non-salt forms of FGF-7 and FGF-7 analogs are encompassed by the scope of the present disclosure.

In some embodiments, the FGF-7 of the present disclosure comprises an FGF-7 analog. In some embodiments, the FGF-7 analog of the present technology is a monomer. In some embodiments, the FGF-7 analog is a non-covalent multimer (e.g., a dimer, tetramer, hexamer, or higher order multimer, a trimer of dimers).

The FGF-7 analogs may be closely related to the structure of human FGF-7, yet contain a modification (e.g. a structural modification) to enhance a certain functional aspect. In some embodiments, the FGF-7 analog may differ from the structure of human FGF-7 by amino acid substitutions only. In some embodiments, the FGF-7 analog may differ from the structure of human FGF-7 by amino acid deletions only. In some embodiments, the FGF-7 analog may differ from the structure of human FGF-7 by amino acid additions only. In some embodiments, the FGF-7 analog comprises a variant or mutant of FGF-7 (e.g., the sequence of SEQ ID NO: 1). In some embodiments, the FGF-7 analog comprises at least 2, at least 3, at least 4, at least 5, at least 6, at least 7, at least 8, at least 9, at least 10, at least 12, at least 15, at least 20, at least 25, at least 30, at least 40, or at least 50 amino acid substitutions, deletions, or additions relative to FGF-7 (e.g., the sequence of FGF-7 as described by SEQ ID NO: 13).

### FGF-7 Fusion Proteins

In a previous disclosure Applicants provided proteins capable of differentiating brown adipocyte progenitors into brown adipocytes. In one aspect, Applicants identified a human protein capable of differentiating brown adipocyte progenitors into brown adipocytes. In another aspect, Applicants identified a human protein capable of differentiating brown adipocyte progenitors isolated from human skeletal muscle into brown adipocytes. Compounds identified in this manner can be used for a variety of research, diagnostic and therapeutic purposes, including, for example, treatment of metabolic diseases such as obesity, type 2 diabetes, insulin-resistance, dyslipidemia, and the like. In some embodiments, an agent identified in the previous disclosure can be optimized for improvement of its physico-chemical and/or pharmacokinetic properties. The present disclosure provides compositions of and methods for such improvement. In some embodiments, such improvement would be represented by an increase in circulating half-life. In other embodiments such improvement would be represented by an increase in the activity of the compound over that of the parent protein. In yet other embodiments such improvement would be represented by an increase in the affinity of the compound for its receptor over that of the parent protein. In still other embodiments, such an improvement would be represented by changes in biodistribution in a way that confers novel biology or improves safety. The present disclosure also provides a method of screening and diagnosing patients and the effectiveness of novel fusion proteins by measuring background UCP1 levels and changes in such UCP1 levels following administration of novel fusion proteins.

In one aspect, the present disclosure provides methods for treating or preventing metabolic disease or conditions in a subject in need thereof comprising administering to the subject an effective amount of the FGF-7-Fc fusion proteins of the present technology. In some aspects these diseases or conditions include, without limitation, obesity, type II diabetes, insulin resistance, hyperinsulinemia, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.

The enhancement of energy expenditure and glucose metabolism by promoting new brown adipocyte formation represents a promising approach for developing new therapeutics for diabetes and obesity. Applicants have demonstrated that EGS0501, an approved molecule, recruits brown adipocytes in vitro and causes significant weight loss (without reducing lean mass) and improvements in insulin resistance and glucose. EGS0501 has a relatively short half-life not suitable for use in obesity, diabetes, and other metabolic conditions. Longer lasting and/or safer analogs of this compound would be desirable and useful, and can be designed, expressed, purified, and tested for therapeutic potential.

In one embodiment, fusion proteins representing analogs of the parent protein can be produced. These include technologies such as fusion to the Fc portion of human immunoglobulins, fusion to human serum albumin (HSA), fusion to human transferrin, XTENylation (also known as rPEG), PASylation, ELPylation, HAPylation, GLK fusion, CTP fusion, or fusion to other compounds such as polyethylene glycol (PEG). In another embodiment a series of Fc-fusions of the protein can be designed, expressed, and purified. This series may include less than 10 or fewer designs, less than 25, 30, 35, 40, 45, 50, 55, 60, 65, 70, 75, 80, 85, 90, 95, or 100 designs, or less than 200, 300, 400, 500, 600, 700, 800, 900, or 1000 designs, or less than 2000, 2500, 3000, 3500, 4000, 4500, or 5000 designs.

The resulting Fc-fusion proteins can be evaluated for their manufacturability and in vitro activity, focusing on those with activity close to that of, equal to, or greater than that of EGS0501. Compounds with sufficient activity can be evaluated in PK studies to understand the potential for significantly extended T_{1/2} in humans. Compounds with the best combinations of manufacturability, in vitro efficacy, and in vivo T_{1/2} can be moved into efficacy studies in the Diet-Induced Obese (DIO) mouse, which is highly predictive of translation into humans. In this way, this process can yield new human FGF-7-Fc fusion proteins showing in vivo efficacy, good manufacturability, and prolonged half-life.

Compositions with higher molecular weight in the circulatory system have a lower propensity to exit the vasculature and enter peripheral tissues. The brown adipocyte progenitor cells previously described by Applicants are found in a pen-vascular location. A strategy of increasing a compound's molecular weight, by Fc fusion or otherwise, would therefore improve the compound's ability to remain within the vasculature where it can recruit new brown adipocytes, but away from parenchymal tissues where it may mediate toxicity. The volume of distribution, or V_{D}, describes a compound's propensity to remain within the vasculature, with a lower V_{D} indicating confinement of a compound to the vascular compartment. The VD of EGS0501 has been reported as 2 L/kg, or approximately 140 L for an average 70 kg human. A strategy of administering a higher molecular weight analog of human FGF-7 could improve V_{D} to about 139 L, 138 L, 137 L 136 L, 135 L, 134 L, 133 L, 132 L, 131 L, 130 L, or to about 125 L, 120 L, 115 L, 110 L, 105 L, 100 L, 95 L, 90 L, 85 L, 80 L, 75 L, 70 L, 65 L, 60 L, 55 L, 50 L, 45 L, 40 L, 35 L, 30 L, 25 L, 20 L, 15 L, 10 L, 9 L, 8 L, 7 L, 6 L, 5 L, 4 L, 3L, 2or 1 L.

Such a strategy would also increase the therapeutic index over that of the parent molecule, by reducing access of compounds to peripheral tissues, and could increase therapeutic index by about 10%, 15%, 20%, 25%, 30%, 35%, 40%, 45%, 50%, 55%, 60%, 65%, 70%, 75%, 80%, 85%, 90%, 95%, or 100%, or by a factor of about 2, 3, 4, 5, 6, 7, 8, 9, 10, or about 15, 20, 30, 40, 50, 60, 70, 80, 90, 100, 200, 300, 400, 500, 600, 700, 800, 900, or 1000 times.

In clinical trials of EGS0501 the most common adverse reactions were skin toxicities (rash, pruritus, erythema, edema), oral toxicities (mouth/tongue thickness or discoloration, and taste disorders), pain, arthralgias, and dysesthesia. A higher molecular weight analog of human FGF-7 that can remain within the vasculature and away from parenchymal tissues where it may mediate toxicity could reduce the incidence of such side effects by about 1%, 2%, 3%, 4%, 5%, 6%, 7%, 8%, 9%, 10%, or by about 15%, 20%, 25%, 30%, 35%, 40%, 45%, 50%, 55%, 60%, 65%, 70%, 75%, 80%, 85%, 90%, 95%, or 100%.

Compounds of higher molecular weight are absorbed more slowly than those of lower molecular weight when administered subcutaneously or via other routes (other than intravenously). This results in a lower maximal concentration (Cmax) being attained. Given that side effects may be wholly or partially a result of a compound's Cmax, a higher molecular weight analog of human FGF-7 such as an Fc fusion could have an improved side effect profile, with the incidence of side effects such as, but not limited to, skin toxicities (rash, pruritus, erythema, edema), oral toxicities (mouth/tongue thickness or discoloration, and taste disorders), pain, arthralgias, and dysesthesias reduced by about 1%, 2%, 3%, 4%, 5%, 6%, 7%, 8%, 9%, 10%, or by about 15%, 20%, 25%, 30%, 35%, 40%, 45%, 50%, 55%, 60%, 65%, 70%, 75%, 80%, 85%, 90%, 95%, or 100%.

In certain embodiments, the FGF-7 polypeptide may be based to the Fc fragment via a peptide linker. Alternatively, no peptide linker may be present between the FGF-7 polypeptide and the Fc domain of the FGF-7-Fc fusion protein (e.g., the C-terminal region of the FGF-7 polypeptide is covalently linked (e.g., via a peptide, bond) to the N-terminal region of the Fc domain or the N-terminal region of the FGF-7 polypeptide is covalently linked via a peptide bond) to the C-terminal region of the Fc domain). Additionally or alternatively, in some embodiments of the FGF-7-Fc fusion protein, the Fc domain comprises a wild-type Fc fragment of human IgG₁, IgG₂, or IgG4.

Peptide linkers may comprise natural or unnatural amino acids. In some embodiments, peptide linkers can be encoded by a nucleic acid molecule, e.g., such that a single nucleic acid molecule can encode the various peptides within an FGF-7 polypeptide as well as the peptide linker(s); or can encode the FGF-7 polypeptide, the Fc fragment, and the peptide linker.

In some embodiments, the peptide linker comprises at least 5 amino acid residues, e.g., at least 5, 6, 7, 8, 9, 10, 11, 12, 13, 14, 15, 16, 17, 18, 19, 20 or more amino acid residues. In some embodiments, the peptide linker comprises 5-9 amino acid residues. In some embodiments, the peptide linker comprises four or less ammo acids or six or more amino acids in length. In some embodiments, the peptide linker comprises 0 amino acids (e.g. no peptide linker). In some embodiments, the peptide linker comprises 4 or more glycines (e.g., 4, 5, 6, 7, 8, or more glycines). In some embodiments, the peptide linker comprises 4 or more consecutive glycines (e.g., 5 or more consecutive glycines). In some embodiments, the peptide linker comprises the amino acid sequence of GGGGAGGGG (SEQ ID NO: 16) or GGGGSGGGG (SEQ ID NO: 17).

Additionally or alternatively, in any of the embodiments herein of the FGF-7-Fc fusion protein, the FGF-7 polypeptide may be located at the N-terminus or C-terminus of the Fc domain.

In some embodiments of the FGF-7-Fc fusion protein, the sequence may include a signal sequence. In some embodiments, an FGF-7-Fc fusion protein described herein does not include a signal sequence at the N-terminus. In other embodiments, an FGF-7-Fc fusion protein described herein includes a signal sequence, e.g., at the N-terminus. An exemplary signal sequence includes the amino acid sequence SEQ ID NO: 14. In some embodiments, an FGF-7-Fc fusion protein described herein is encoded by a nucleic acid molecule comprising a signal sequence, e.g., for expression (e.g., recombinant expression) in cells (e.g., eukaryotic, e.g., mammalian cells). In certain embodiments, the signal sequence is cleaved off, e.g., in the cell culture, during expression. An exemplary nucleic acid sequence encoding a signal sequence includes the nucleic add sequence SEQ ID NO: 15. In other embodiments, a fusion protein described herein is encoded by a nucleic acid molecule not comprising a signal sequence.

The present disclosure also provides kits for the prevention and/or treatment of a metabolic disease (e.g., obesity, e.g., Type 2 diabetes) comprising one or more of the FGF-7-Fc fusion proteins described herein. Optionally, the above described components of the kits of the present technology are packed in suitable containers and labeled for the prevention and/or treatment of a metabolic disease (e.g., obesity, e.g., Type 2 diabetes).

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic products, that contain information about, for example, the indications, usage, dosage, manufacture, administration, contraindications and/or warnings concerning the use of such therapeutic or products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

### Example 1

Creation of novel human FGF-7-Fc fusion proteins and evaluation of yield: The properties of fusion proteins can vary widely versus the underlying protein depending on many factors, including the orientation of the protein of interest, fusion to the N-terminal or C-terminal of the Fc moiety, the nature of the linker sequence, oligomerization of the fusion protein, and other features. In addition, a traditional Fc fusion, like a full antibody, has two chains linked by a disulfide bond and contains two copies of the underlying protein. Monomers of the fusion can be created in which only a single copy of the underlying protein is present. In fact, monomerization has been shown to increase physiologic uptake, bioavailability, and the serum half-life of fusion proteins (Dumont et al., 2006).

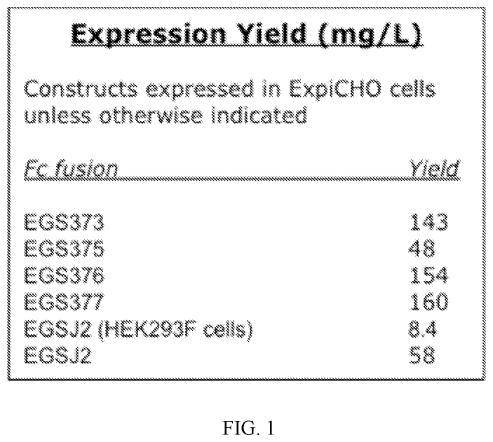

Initially, diverse DNA constructs corresponding to human FGF-7-Fc fusion proteins were designed according to established principles of fusion protein engineering and development. Variants were, for example, based on the site of fusion, the length and sequence of the linker, and the orientation of the protein vs the Fc moiety. Chinese Hampster Ovary (CHO) cells were then transiently transfected. Yields can vary significantly depending on structure, and the determination of yield for a given construct is largely empirical. Yield was first evaluated in pilot scale fermentations, and clones yielding≥5 mg protein from 1 L cultures were taken forward. Multiple rounds of iterative fusion design and testing were performed. Demonstration of feasible yield (i.e., manufacturability) is a critical early risk-reduction step in selecting fusion protein constructs that can be produced at sufficient scale in a research cell bank setting and scaled up for master cell bank production to prepare quantities of these proteins for clinical testing in man and commercialization (FIG. 1).

Various procedures may be used for the production of the FGF-7-Fc fusion proteins described herein. (See, for example, Antibodies: A Laboratory Manual, Harlow E. and Lane D, 1988, Cold Spring Harbor Laboratory Press, Cold Spring Harbor, N.Y., incorporated herein by reference).

Vectors. An FGF-7-Fc fusion protein can be expressed by a vector comprising any of the DNA sequences encoding an FGF-7-Fc fusion protein of the present technology as described herein. These can include nucleic acid vectors, liposomes, naked DNA, adjuvant assisted DNA, gene gun, catheters, etc. Vectors can include chemical conjugates such as described in WO 93/64701, which has targeting moiety (e.g. a ligand to a cellular surface receptor) and a nucleic acid binding moiety (e.g. polylysine), viral vectors (e.g. a DNA or RNA viral vectors), plasmids, phages, etc. The vectors can be chromosomal, non-chromosomal or synthetic.

Exemplary vectors include viral vectors, fusion proteins and chemical conjugates. Retroviral vectors include moloney murine leukemia viruses. In some embodiments, the viral vector is a DNA viral vector. Exemplary DNA vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: USA. 90:7603 (1993); Geller, A. I., et al., Proc Natl. Acad. Sci. USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplin, M. G. et al., Nat. Genet. 8:148 (1994).

Pox viral vectors introduce the gene into the cell cytoplasm. Avipox virus vectors result in only a short term expression of the nucleic acid. In some embodiments, adenovirus vectors, adeno-associated virus vectors and herpes simplex virus (HSV) vectors are used for introducing the nucleic acid into cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors. These vectors can be used to express the FGF-7-Fc fusion proteins described herein.

Cell Lines, Expression and Purification: Also disclosed herein are host cells that express FGF-7-Fc fusion protein of the present technology or a vector comprising any of the DNA sequences encoding an FGF-7-Fc fusion protein of the present technology.

In some embodiments, an FGF-7-Fc fusion protein can be expressed recombinantly, e.g., in a eukaryotic cell, e.g., mammalian cell or non-mammalian cell. Exemplary, mammalian cells used for expression include HEK cells, e.g., HEK293 cells, or CHO cells, among other cell lines available in the art, e.g., cell lines used for expression of antibody fragments or Fc containing proteins. In some embodiments, non-mammalian cells, such as insect cells are used for expression of the FGF-7-Fc fusion proteins of the present technology, e.g., SF9 or S2 cells, among other cell lines available in the art, e.g., cell lines used for expression of antibody fragments or Fc containing proteins. In some embodiments, cells are transfected with a nucleic acid molecule, e.g., vector, encoding the FGF-7-Fc fusion protein (e.g., where the entire FGF-7-Fc fusion protein is encoded by a single nucleic acid molecule). In other embodiments, cells are transfected with more than one nucleic acid molecule, where each nucleic acid molecule encodes a different domain of the FGF-7-Fc fusion protein. For example, one nucleic acid molecule can encode the FGF-7 polypeptide, and a different nucleic acid molecule can encode the Fc fragment. Cells can be cultured using standard methods in the art.

In some embodiments, the FGF-7-Fc fusion protein is purified or isolated from the cells (e.g., by lysis of the cells). In other embodiments, the FGF-7-Fc fusion protein is secreted by the cells and, e.g., the fusion protein is purified or isolated from the cell culture media in which the cells were grown. Purification of the FGF-7-Fc fusion protein can include using column chromatography, e.g., affinity chromatography, or using other separation methods that involve size, charge, and/or affinity for certain molecules. In some embodiments, purification of the FGF-7-Fc fusion protein involves selecting/enriching for proteins with an Fc fragment, e.g., by using Protein A beads or a Protein A column. Other affinity separation methods can be used, e.g., using anti-FGF-7 antibodies or fragments thereof. Additionally or alternatively, other separation methods such as ion exchange chromatography and/or gel filtration chromatography can also be employed. In some embodiments, purification of the FGF-7-Fc fusion protein further comprises filtering or centrifuging the protein preparation.

The purified fusion protein can be characterized, e.g., for purity, yield, structure, and/or activity, using a variety of methods, e.g., absorbance at 280 nm (e.g., to determine yield), size exclusion or capillary electrophoresis (e.g., to determine the molecular weight and/or purity), mass spectrometry (MS) and/or liquid chromatography (LC)-MS (e.g., to determine purity and/or glycosylation), and/or ELISA (e.g., to determine extent of binding, e.g., affinity, to an anti-FGF-7 antibody). Exemplary methods of characterization are also described in the Examples section.

In some embodiments, expression of an FGF-7-Fc fusion protein in a cell, e.g., cell culture, generates a yield of at least 5 mg of the FGF-7-Fc fusion protein (e.g., purified fusion protein) per liter of culture (e.g., at least 5 mg/L, 10 mg/L, 15 mg/L, 20 mg/L, 25 mg/L, 30 mg/L, 35 mg/L, 40 mg/L, 45 mg/L, 50 mg/L, 60 mg/L, 70 mg/L, 80 mg/L, 90 mg/L, 100 mg/L, 110 mg/L, 120 mg/L, or more). In sonic embodiments, a purified FGF-7-Fc fusion protein has a purity of at least 80% (e.g., at least 80%, 85%, 90%, 95%, 97%, 99% by weight), e.g., as determined by standard methods.

### Example 2

Evaluation for aggregation: Aggregation of Fc fusion proteins has consequences for decreasing production, lowering bioactivity and serum half-life, and has been implicated in immunogenicity. The mechanisms of protein aggregation vary depending on the protein, linker, and the expression system utilized, and it is difficult a priori to predict which hypothetical Fc fusions of a particular protein will exhibit unacceptable aggregation into high molecular weight species. Aggregation is a complex phenomenon and no single analytical method is optimal for all types and sizes of aggregates. Techniques to study the degree of aggregation of expressed Fc fusion proteins may be used, which may include any combination of size exclusion chromatography, multi-angle classical laser light scattering used on-line with SEC (SEC-MALLS), sedimentation velocity, field-flow fractionation, and analytical ultracentrifugation.

### Example 3

Confirmation of activity in cellular model: Those fusion proteins with good potential manufacturability were tested for in vitro activity versus EGS0501. The agents were tested in a broad concentration range (100 pM to 1 μM), and those analogs showing in vitro activity at least 75% that of the maximal effect observed with EGS0501 at sub-micromolar concentrations (EC50≤100 nM) were selected for further study. A growth assay was used to assess activity of the Fc fusions. Rhesus monkey bronchial lung epithelial cells (4MBr-5, CCL208; American Type Culture Collection, Manassas, VA) were used as they are known to be very sensitive to FGF-7 as well as Epidermal Growth Factor (EGF) (Rubin et al., 1989). The cells were cultured in F-12K media (American Type Culture Collection) with 10% Fetal Bovine Serum (FBS) (Gibco), and 30 ng/ml EGF (Sigma #E4127). The media was changed every 2 to 3 days and the cells were passaged at 1:2 or 1:4 when they reached 65-75% confluency.

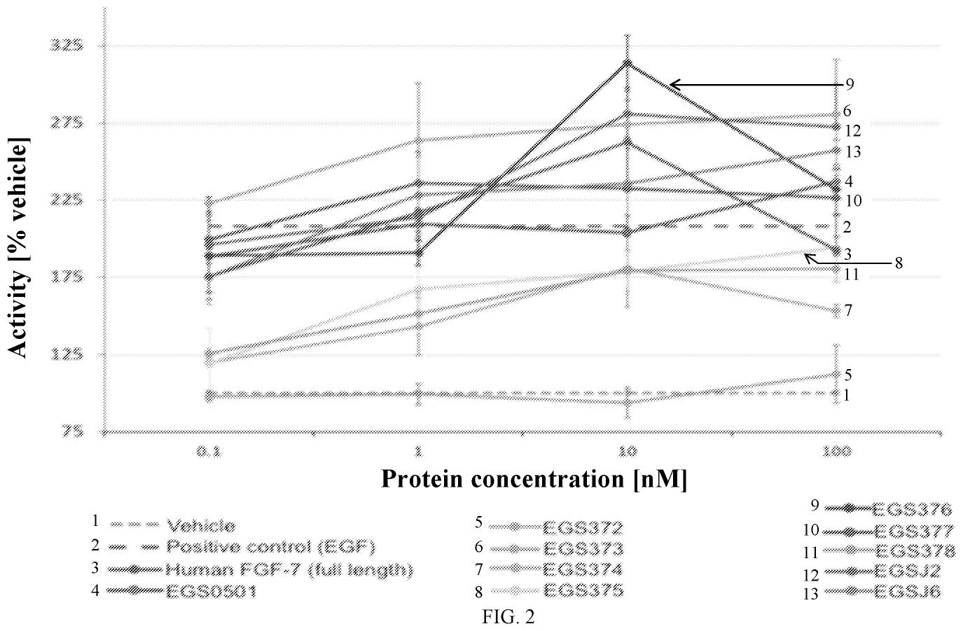

Briefly, for the growth assay tissue culture-treated 96-well plates were used. The 4MBr-5 cells were trypsinized and suspended in F-12K media with 2.5% FBS (assay medium) or 10% FBS (regular medium). Wells containing 2.5% FBS and 10% FBS +/−EGF (30 ng/ml) were used as controls. A dose response of hFGF7 (Biovision #6450) was also run as a reference for the human FGF-7-Fc fusion proteins. Dilutions of the fusion proteins and controls were made in triplicate directly in the 96 well plates, followed by the addition of 10,000 4MBr-5 cells per well. On day 5, cell number was measured by total dsDNA using Hoechst DNA quantitation (ThermoFisher) according to the manufacturer's protocol (FIG. 2).

Human FGF-7-Fc fusion proteins with a combination of high in vitro activity and sufficient production yield to support development were selected for PK studies in mice.

### Example 4

Selecting analogs of human FGF-7 with improved in vivo half-life (T_{1/2}): Studying the pharmacokinetics (PK) of each compound in mice permits the selection of those with plasma half-lives significantly longer than that of EGS0501 itself, which is 4-5 hours. A threshold of ≥3×half-life can be used, although other thresholds may be set as well including for example 24 hours, 48 hours, 72 hours, 96 hours, 5 days, 6 days, 7 days, 10 days, 14 days, or more. It is possible that half-lives much longer than 3×the half-life of EGS0501 can be observed, as the T_{1/2} of Fc-fusion proteins is often 4-5 days and sometimes a week or more (Kuo et al., 2010). However, mice clear many human Fc fusions more rapidly, and in order not to exclude potentially promising compounds that may have much longer half-lives in non-human primates and humans, a threshold of 6-9 hours is appropriate at this stage.

C57B1/6 male mice aged 8 weeks were used. Compounds were dissolved in vehicle (in one embodiment, 9.6 mg/ml mannitol, 4.8 mg/ml sucrose, 0.37 mg/ml L-histidine, 0.025 mg/ml polysorbate 20, pH adjusted to 7.4 with HCl in water) and injected either IV (tail vein) or IP at 0.3-3 mg/kg. A number of mice were used per time point, and 50 μL of blood was collected at 1, 3, 6, 12, 24, 48, and 72 hours, and 7 and 14 days following administration. A total of ≤150 μL of blood (4 time points maximum) was collected from individual mice. Blood samples (in EDTA tubes) were kept on ice, centrifuged (5000×g, 5 minutes), and plasma isolated and frozen at −80° C. until analysis of compound levels was performed.

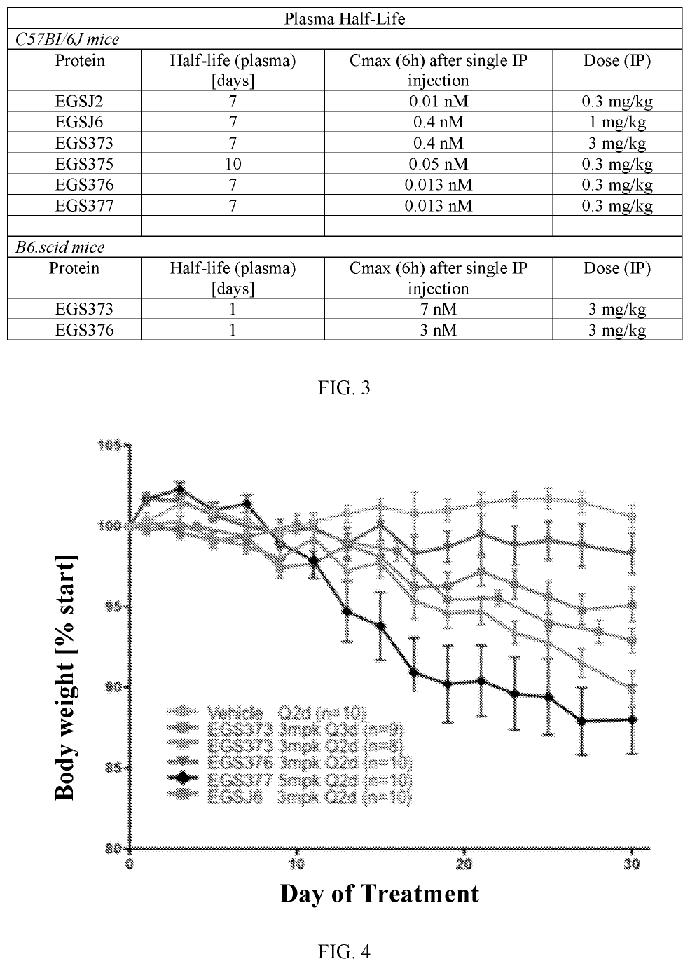

The concentrations of each of the human Fc fusions at each time point were quantified with a bi-specific ELISA using antibodies specific for human IgG and human FGF-7. Briefly, 96 well Nunc MaxiSorp flat bottom plates (Invitrogen #44-2404-21) were coated at 4° C. overnight with 1.5 ug/ml anti-hFGF7 (Clone #29522, R&D MAB251) in PBS. After washing the plate (PBS+0.05% Tween20) the wells were blocked for 2 hours at room temperature with blocking buffer (ThermoFisher #37573). After washing, plasma samples and standards were diluted in assay buffer (Invitrogen D598200) and added in duplicate to the assay plate. Plasma samples were typically diluted 1:4. After 2 hours at room temperature the plate was washed and a polyclonal peroxidase-conjugated anti-human IgG (1:40,000 in assay buffer) was added for 1 hour at room temperature. The plate was washed and 3,3′,5,5′-tetramethylbenzidine (Sigma-Aldrich #T0440) added. After 20 minutes the reaction was stopped (ThermoFisher #N600). Absorbance at 450 nm was read on a Varioscan plate reader (FIG. 3).

At the conclusion of Example 4 a number of human FGF-7-Fc fusions with the greatest in vivo plasma half-life in normal mice were selected for efficacy studies in a mouse model of human obesity and insulin resistance. As noted previously, as the properties of Fc fusions are difficult to predict based on their sequence, synthesis of a diverse collection of variants at the outset increases the likelihood that at least some of the fusions will exhibit prolonged T_{1/2} in mice.

### Example 5

Identifying human FGF-7-Fc fusion proteins with prolonged T_{1/2} that reduce body weight by at least 10% in Diet-Induced Obese (DIO) mice after 28 days of dosing.

The therapeutic efficacy of a number of selected human FGF-7-Fc fusion proteins with the longest plasma half-life was evaluated. Diet-Induced Obesity (DIO) in mice is a well-established model of obesity and insulin resistance and was used in these experiments.

Brown adipocyte recruitment is known to require a chronic stimulus and longer-term dosing (of at least 10-14 days, preferably 28 days or more) is desired to evaluate in vivo efficacy. Applicants previously demonstrated that this length of dosing is sufficient to demonstrate robust effects using EGS0501 in several animal models of obesity, including DIO mice.

The tested human FGF-7-Fc fusions are based on human protein sequences. They therefore have a high probability of eliciting an immune response in mice, which may include the production of neutralizing antibodies. While this is generally not operative with a single dose as in single-dose PK studies, repeat dosing, as required in longer term efficacy studies, is more likely to generate an immune response in the subject animal. An immune-compromised model was therefore developed for these experiments. B6.Cg-Prkdc^{scid}/SzJ mice (Jackson Laboratories, Bar Harbor, ME), also known as “C57B1/6 scid” mice, carry the severe combined immune deficiency mutation (scid, which is caused by a spontaneous mutation in the Prkdc gene) on the C57BL/6J strain background. The mutant mice do not have functional T or B cells, thus supporting the introduction of foreign proteins. These mice were fed a high fat diet, and were demonstrated to readily become obese, reaching weights of up to 50 grams.

Fc fusions, like intact immunoglobulins, are recycled intracellularly via the FcRn receptor. The lack of B cells and antibodies in scid mice however means that the FcRn receptor is unoccupied in these animals. This leads to the possibility that the PK, and specifically the circulating half-life of Fc fusions could be different in scid mice versus non-scid animals. The half-life of the various human FGF-7-Fc fusions was therefore determined in lean C57B1/6 scid mice, in order to provide for an appropriate dosing interval in the efficacy studies. The same techniques as those described above were used (FIG. 3).

C57B1/6 scid male mice (B6.scid, The Jackson Laboratory, Bar Harbor, ME, USA, stock no. 001913), aged 2 months, were fed a high fat (60% calories) diet (Research Diets D12492) for 8 to 12 weeks. The animals were maintained for at least 2 weeks prior to the dosing period as well as during the study at a temperature close to thermoneutrality for mice (28-30° C.). Under these conditions, existing brown adipose atrophies (Boss et al., 1988) and mice with a C57B1/6 background develop obesity, insulin resistance, and impaired glucose tolerance, with low to moderate hyperglycemia (diabetes). Only animals weighing≥40 g were used in the study to provide an adequate window to observe weight loss of 10% or more. Stratified randomization was employed to ensure that animals of similar weights were distributed across the study groups at the start of the study.

The human FGF-7-Fc fusion proteins selected for in vivo efficacy testing were expressed at higher scale, purified, and dissolved in vehicle. As a run-in to the study, mice were dosed with saline by intraperitoneal (IP) injection for 3 days once daily. Mice were subsequently dosed by IP injection over 28 days, with the study including negative and positive control groups, receiving vehicle alone or EGS0501, respectively. Dosing levels and frequency of administration (every 2 or 3 days) for each compound were based on the results from the in vitro activity and in vivo PK studies. Body weight was measured at baseline, 2×per week, and at the end of the study. Samples for fasted (6 hours) plasma glucose, insulin , and leptin were obtained at baseline and at the end of the study. Insulin sensitivity was determined using the homeostasis model assessment of insulin resistance (HOMA-IR) (HOMA-IR=(plasma insulin [microIU/ml]×plasma glucose [mM])/22.5). Food intake was monitored by weighing the food given and remaining in the cages on a per cage (4 mice) basis. Body composition was assessed at the end of the study with EchoMRI. At the conclusion of the dosing period, half of the mice in two groups (EGS373 dosed every 3 days, and vehicle dosed every 2 days) were allowed to recover, without further dosing, until their body weight returned to baseline.

Methods

Animal Studies

During the dosing period mice were dosed every 2 or 3 days by intraperitoneal (IP) injection (100 μl per mouse) with either vehicle alone (9.6 mg/ml mannitol, 4.8 mg/ml sucrose, 0.37 mg/ml L-histidine, 0.025 mg/ml polysorbate 20 (Tween 20, CAS#9005-64-5), pH adjusted to 7.4 with HCl) or test article dissolved in vehicle, for 28 days.

During the study food intake was measured by manually weighing the food in the hopper and any pieces left at the bottom of the cage on a daily basis.

Nine days prior to the start (d-9) and at the end of the dosing period (d29), as well as at days 55 and 76 for the animals kept for recovery, mice were fasted for 6 hours, and blood was collected. Plasma was isolated and saved at −20° C. until use for measurement of glucose, insulin, and leptin levels (University of Cincinnati Mouse Metabolic Phenotyping Center). At the end of the study the epididymal adipose depot (the major white fat pad in mice) was collected and weighed. The weight of this depot, as well as the plasma leptin levels, can be used as indices of whole-body fat content.

All blood glucose measurements were made by glucometer using blood obtained via venipuncture of a peripheral blood vessel (i.e., tail vein). A maximum of 150 μl of blood was taken from each mouse per time point in order to avoid inducing anemia in the test animals. Samples were placed on ice immediately following collection, and plasma isolated by centrifugation (5000×g, 5 minutes) within 30 minutes of bleeding time. Samples were frozen until further analysis was performed. At the last time point animals (other than those in the recovery groups) were euthanized by CO₂ asphyxiation and cervical dislocation, and blood was collected by cardiac puncture. Plasma insulin, glucose, and leptin levels were measured on terminal plasma samples by ELISA (Lee et al., 2014) (University of Cincinnati Mouse Metabolic Phenotyping Center). Plasma levels of the compounds were measured on the blood collected by cardiac puncture. The plasma concentrations of the compounds were quantified by bi-specific ELISA, as above.

Following terminal bleed, tissues may be collected within 10 minutes of sacrifice, frozen in liquid N₂ and stored at −80° C. In one embodiment these include interscapular BAT, gonadal WAT, subcutaneous WAT from the back and inguinal depots, gastrocnemius and spinotrapezius muscles, and liver. In addition, prior to being frozen the gonadal WAT, inguinal WAT, and interscapular BAT (after careful removal of surrounding white adipose tissue) can be weighed. The weight of the gonadal and inguinal WAT depots can be used to confirm the effect of compound treatment on body fat (as measured by EchoMRI), as these depots are the two major white fat stores in terms of size, and therefore serve as a robust indicator of total body fat content in DIO mice (Luu et al., 2008). Total energy expenditure (relative values for compounds vs. vehicle) may also be calculated from body weight, body composition, and cumulative food intake. An initial evaluation of bone density can be made using Dual Energy Xray Absorptiometry (DEXA).

The thermogenic capacity of the interscapular BAT depot is largely reflected by the weight of the tissue and the level of UCP1 expression. UCP1 expression may be measured by TaqMan real-time PCR for UCP1 mRNA, as described above and UCP1 can be measured in all adipose and skeletal muscle tissues collected. UCP1 gene expression has been shown to be mainly regulated at the transcriptional level, and changes in mRNA levels are largely reflected by changes in UCP1 protein. Additional mouse brown adipocyte markers in various tissues may also be quantified to corroborate the UCP1 findings, including Cidea (Cell death activator CIDE-A), a gene highly enriched in brown adipocytes, Elovl3 (Elongation of very long chain fatty acids protein 3, Cig30), also highly enriched in brown adipocytes, COX IV (Cytochrome c oxidase subunit 4 isoform 1, mitochondrial), a gene which reflects tissue mitochondrial density and is more abundant in brown than in white adipose tissue, and FGF21 (Kolumam et al., 2015). In some samples the expression of the general adipogenic marker PPARγ2, and leptin, which reflects the mass of white adipose tissue, may also be quantified. Cyclophilin A, a housekeeping gene, is typically also measured to reflect the total amount of RNA/cell number.

Data Analysis

Data from in vivo mouse studies are presented as mean±SEM. Significances were evaluated using unpaired Student's t-test, 2-way ANOVA or 1-way ANOVA with Bonferroni's or Dunnett's multiple comparison test using GraphPad Prism version 7 or 8 (GraphPad Software, San Diego, CA). Significances were set at p<0.05, with *: p<0.05 vs. Vehicle, **: p<0.01 vs. Vehicle, ***: p<0.001 vs. Vehicle.

Results

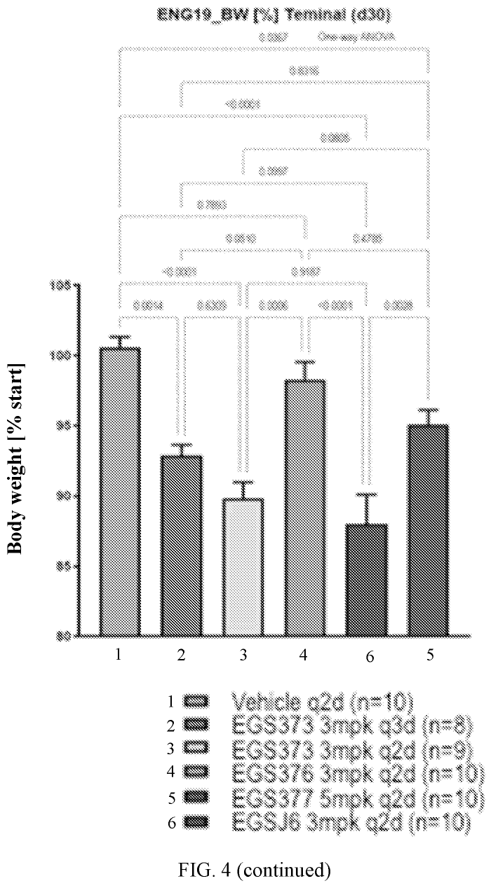

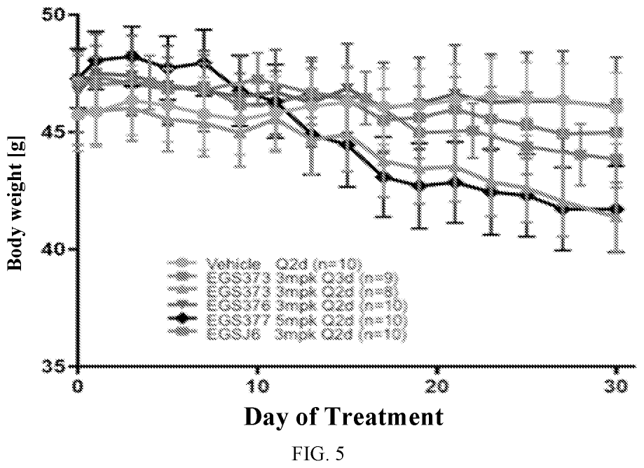

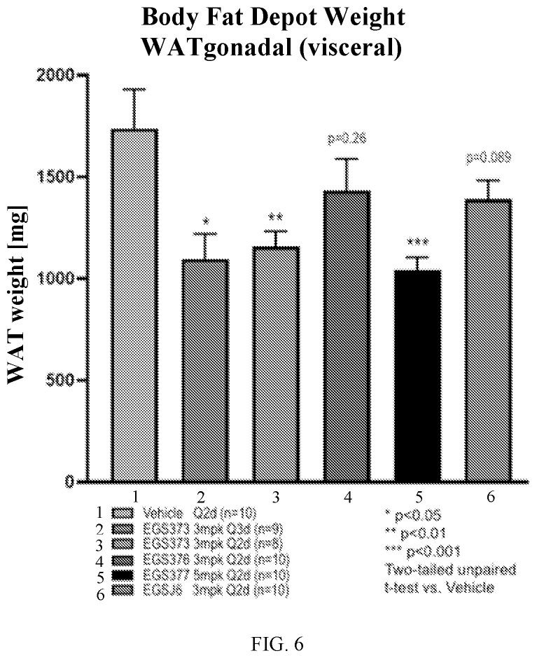

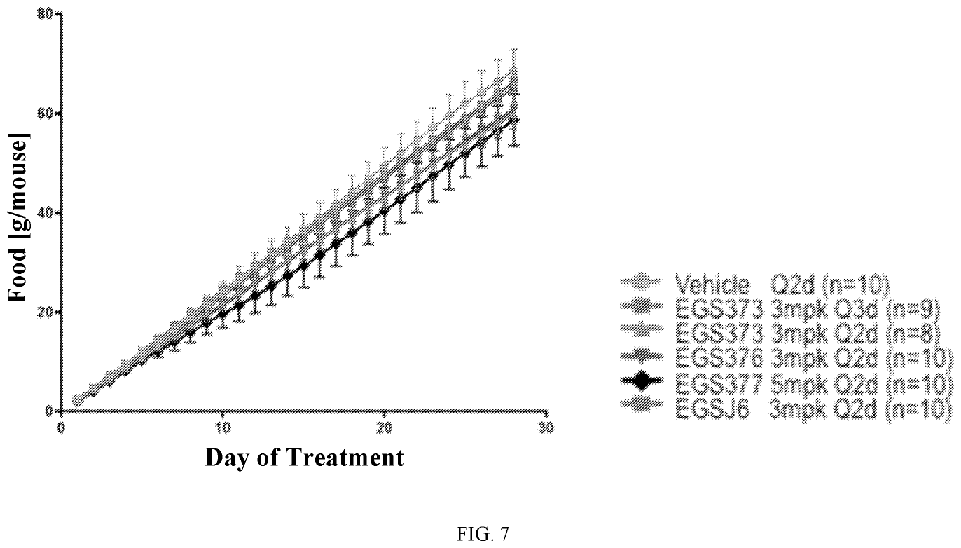

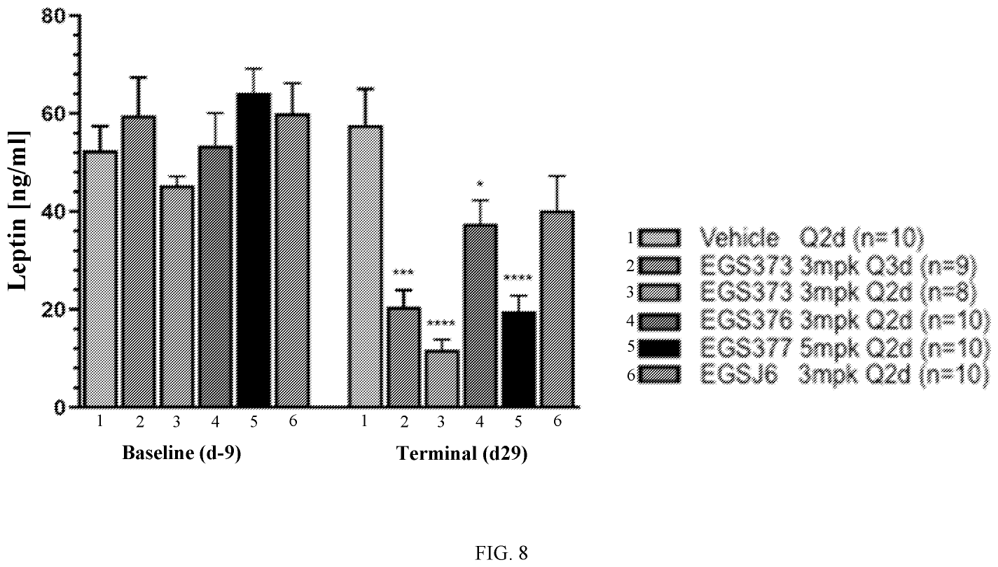

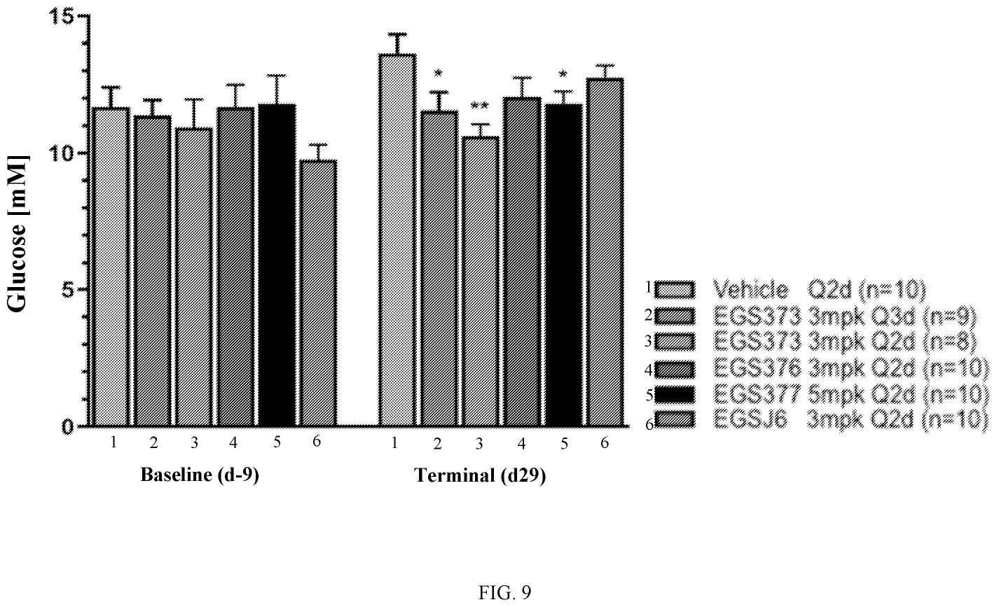

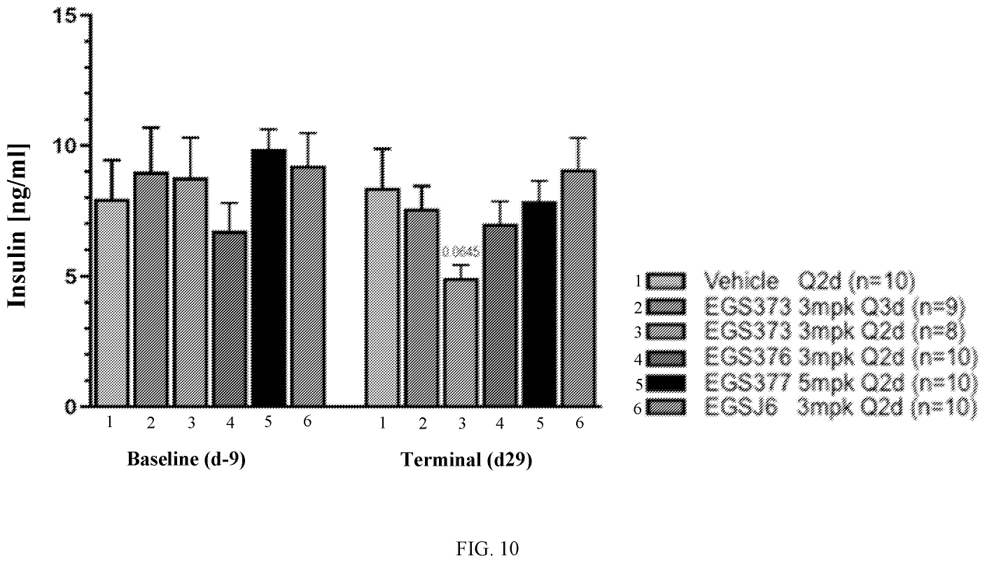

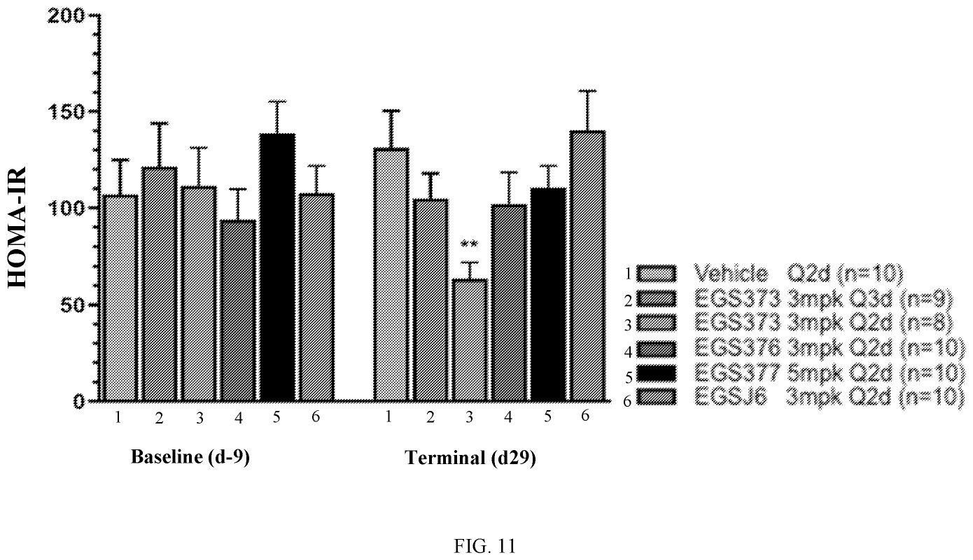

EGS373, EGS377 and EGSJ6 were found to induce significant body weight loss after 28 days of dosing (FIG. 4, percent of starting weight, and FIG. 5, grams). Epididymal (visceral) fat content was similarly reduced (FIG. 6). There was no significant effect on food intake (FIG. 7). This was accompanied by reductions in plasma levels of leptin (FIG. 8) and glucose (FIG. 9). Plasma levels of insulin (trend) (FIG. 10) and the index of insulin resistance, HOMA-IR (FIG. 11) were reduced by EGS373 given every 2 days.

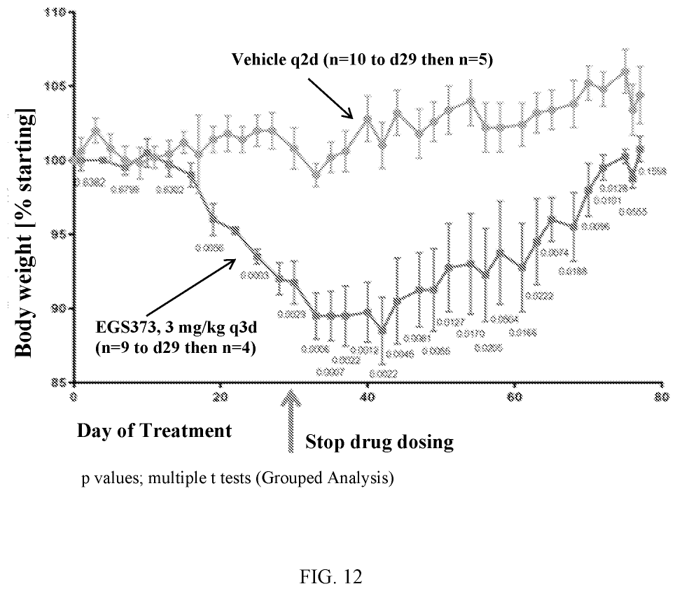

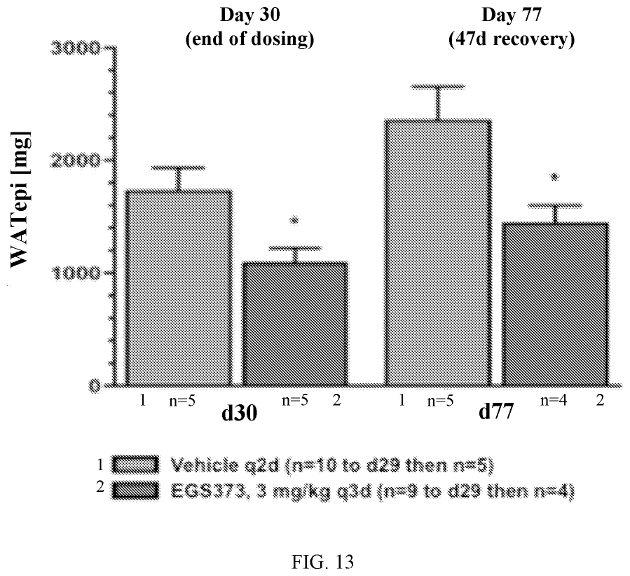

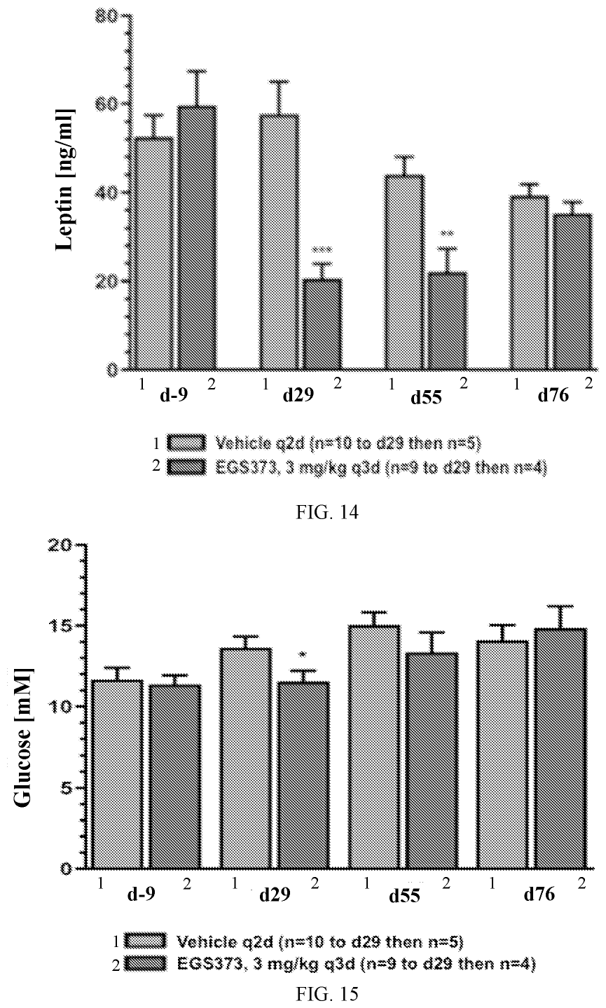

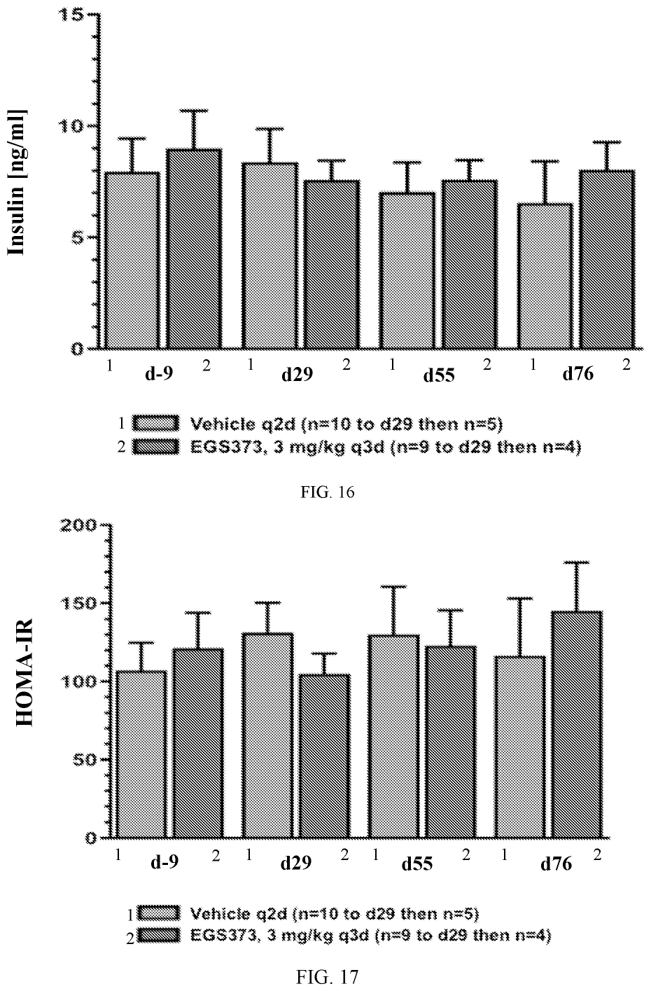

Recovery following 28 days of dosing with EGS373: Mice that were dosed with EGS373 every 3 days for 28 days were maintained in their cages post-dosing and with continued access to the high fat diet. The mice progressively regained body weight and reached a mean weight not statistically different from their baseline at day 77 of the study (FIG. 12). The epididymal fat pad weights had not fully returned to baseline at day 77 (FIG. 13), while the plasma leptin levels were similar to the levels seen in vehicle-treated mice (FIG. 14). Plasma levels of glucose (FIG. 15) also returned to the levels seen in vehicle-treated mice. The values for insulin (FIG. 16) and HOMA-IR (FIG. 17) over the course of the study are also shown. While the latter do not show a significant difference vs vehicle, the values for the EGS373-treated group relative to those for the vehicle group over time suggest an effect on these parameters.

The human FGF-7-Fc fusions with the most favorable profiles in terms of body weight loss and improvement in insulin sensitivity over 28 days of dosing, as well as the best combination of potency and dosing frequency (lowest dose and least frequent dosing) were identified (SEQ ID NO: 2 to SEQ ID NO: 7). These compositions could not have been identified a priori as the properties of such sequences cannot be predicted with any reasonable accuracy by those skilled in the art.

Subsequently, Research Cell Bank (RCB) development, formulation development, reduction of any significant aggregation, and immunogenicity studies in Non-Human Primates may be performed, leading to selection of a lead compound(s).

Unless otherwise defined, all technical and scientific teens used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

### Example 6

A human FGF-7-Fc fusion protein increases energy expenditure and improves metabolic health in a mouse model of obesity and diabetes (B6.scid Diet-Induced Obese (DIO) mice) after 28 days of dosing

Obesity and insulin resistance, an early stage in the development of type 2 diabetes, was induced in B6.scid mice by feeding the mice with a high fat diet (Research diet, Cat #D12492, 60% fat kcal) for 12 weeks. After the phenotype was established, mice were dosed with the compound of interest (EGS373) or vehicle for 28 days. The day after the last dose, animals were fasted for six hours and euthanized by CO₂. Blood was collected, and plasma was used to measure insulin, glucose and leptin levels. Insulin sensitivity was determined using the homeostasis model assessment of insulin resistance (HOMA-IR).

Methods

Animal studies

Diet-induced obese scid mice: C57B1/6 scid (B6.scid, The Jackson Laboratory, Bar Harbor, ME, USA, stock no. 001913) male mice were fed with a high fat diet (Research diet, Cat #D12492, 60% fat kcal) for 12 weeks starting at 6 weeks of age. The mice were maintained at 22-23° C. with abundant bedding material, maintaining the animals in an environmental temperature close to their thermoneutrality, starting 2 weeks prior to the dosing period and for the full dosing period, with a 12 h/12 h light/dark cycle. Under these conditions, existing brown adipose atrophies and mice with a C57B1/6 background develop obesity, insulin resistance, and impaired glucose tolerance, with low to moderate hyperglycemia (diabetes). Only animals weighing≥40 g were used in the study to provide an adequate window to observe weight loss of 10% or more. Stratified randomization was employed to ensure that animals of similar weights were distributed across the study groups at the start of the study.

Two days before the start and at the end of the dosing period, animals were fasted for 6 hours, and blood was collected. Plasma was isolated and saved at −20° C. until used for measurement of glucose, insulin and leptin levels (University of Cincinnati Mouse Metabolic Phenotyping Center).

During the dosing period mice were dosed every 3 days by intraperitoneal (IP) injection (100 μl per mouse) with either vehicle alone (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or the test article (3 mg/kg) dissolved in vehicle, for 28 days.

After 25 days of dosing (9 doses) mice were transferred, individually, to calorimetric chambers (Comprehensive Lab Animal Monitoring System (CLAMS), Columbus Instruments, Columbus, OH) for measurement of energy expenditure via indirect calorimetry, food intake, and locomotor and ambulatory activity for 3 days, including one day for assessment of 24-h energy expenditure and one day for assessment of maximal thermogenic capacity (Beth Israel Deaconess Medical Center Metabolic Core, Boston, MA). Maximal thermogenic capacity, reflecting whole-body brown/brite/beige adipose mass, was assessed by injecting the mouse β3-adrenergic agonist CL316243 (1 mg/kg IP) and measuring metabolic parameters (energy expenditure) over 5 hours post-injection.

Statistical Analysis

Data from in vivo mouse studies are presented as mean±SEM. Significances were evaluated using unpaired Student's t-test, 2-way ANOVA or 1-way ANOVA with Bonferroni's or Dunnett's multiple comparison test using GraphPad Prism version 7 or 8 (GraphPad Software, San Diego, CA). Significances were set at p<0.05, with *: p<0.05 vs. Vehicle, **: p<0.01 vs. Vehicle, ***: p<0.001 vs. Vehicle.

Results

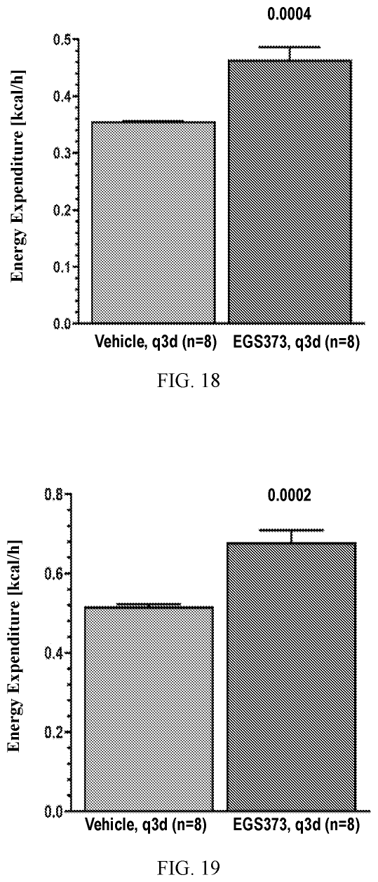

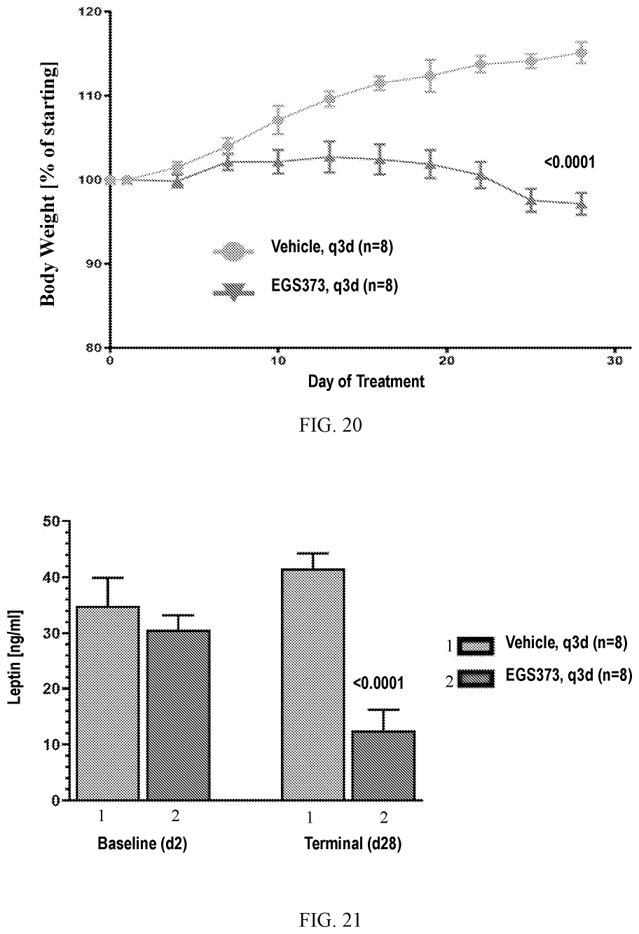

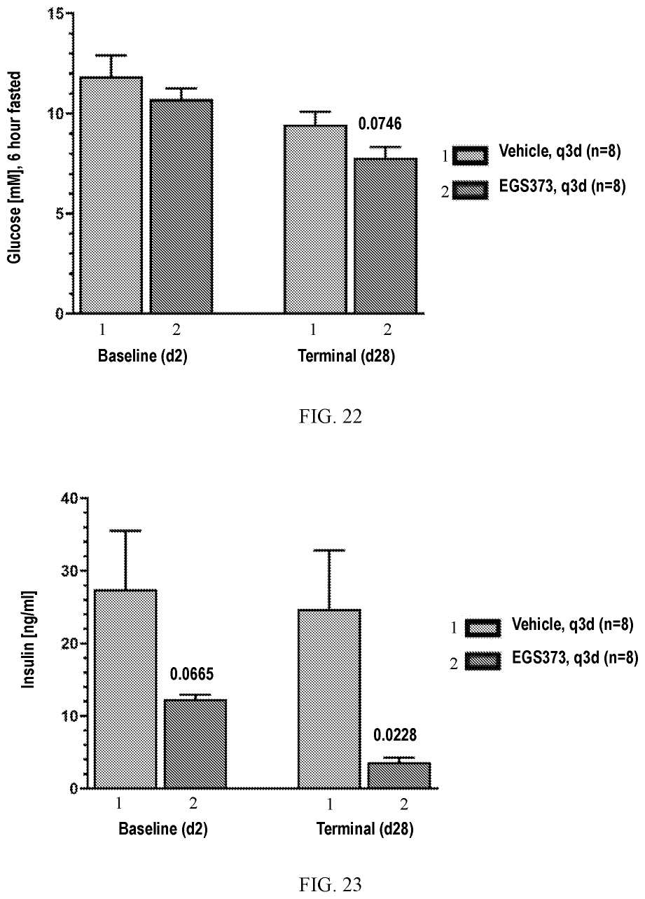

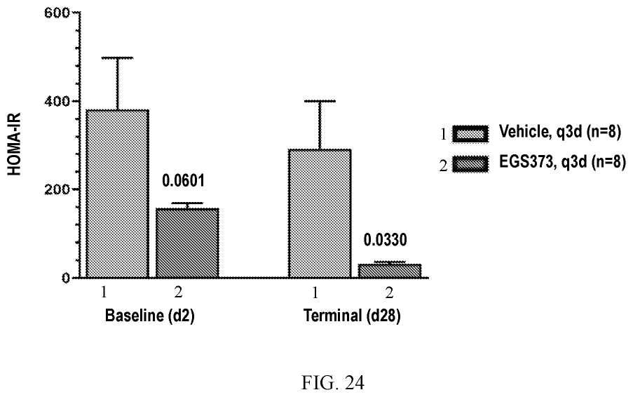

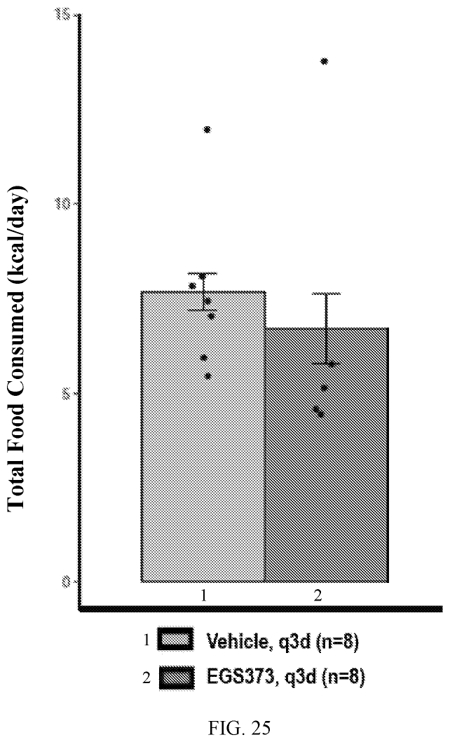

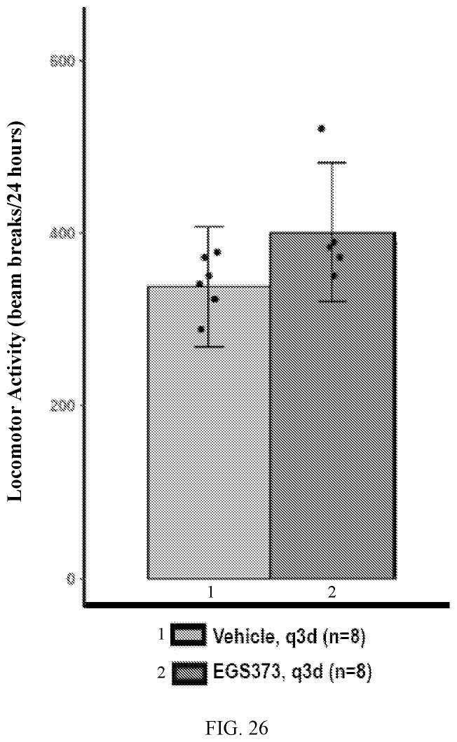

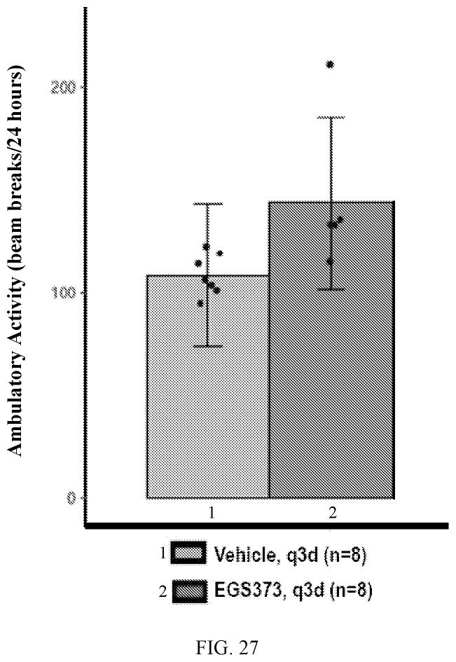

EGS373 was found to increase resting energy expenditure (FIG. 18), and increase the amount of brown fat, as indicated by the thermogenic response to CL316243 (FIG. 19) in the mice. This was accompanied by improvement in parameters of metabolic health: decreased body weight (FIG. 20), plasma levels of leptin (FIG. 21), glucose (trend) (FIG. 22), insulin (FIG. 23), and an index of insulin resistance (HOMA-IR) (FIG. 24). These favorable metabolic effects were observed in the absence of effects of EGS373 on food intake (FIG. 25) or locomotor (FIG. 26) or ambulatory activities (FIG. 27).

In FIGS. 18-24, EGS373 (3 mg/kg every 3 days, intraperitoneal injection) or vehicle (every 3 days, intraperitoneal injection) was dosed for 25 days in B6.scid DIO mice. Throughout the study the mice were maintained on the high fat diet. After 25 days of dosing (9 doses) mice were transferred, individually, to calorimetric chambers (CLAMS) for measurement of energy expenditure by indirect calorimetry, food intake, and locomotor and ambulatory activity for 3 days, including one day for assessment of 24-h energy expenditure and one day for assessment of maximal thermogenic capacity. Plasma parameters were measured at baseline (2 days prior to dosing period) and at the end of the dosing period (day 28), following a 6 hour fast.

### Example 7

A human FGF-7-Fc fusion protein induces body weight loss and beneficial effects on glucose handling without effects on food intake or inducing pica in a mouse model of obesity and diabetes

Obesity and insulin resistance, an early stage in the development of type 2 diabetes, was induced in B6.scid mice by feeding the mice a high fat diet. After the phenotype was established, mice were dosed with compound or vehicle once every 3 days by intraperitoneal (IP) injection for 28 days. The day after the last dose, the mice were fasted for six hours and euthanized by CO₂. Blood was collected, and plasma was used to measure insulin, glucose and leptin levels. Insulin sensitivity was determined using the homeostasis model assessment of insulin resistance (HOMA-IR).

Rodents physiologically cannot exhibit emesis. Rather, following consumption of what would be a nausea-inducing substance in humans, rodents exhibit pica, which is the consumption of non-food substances such as clay (kaolin). Pica studies are routinely used to predict whether a compound may induce nausea in humans and may in fact produce weight loss, either in whole or in part, by inhibiting food intake. At the outset of this study, 5 mice were dosed with a single injection of cisplatin (4 mg/kg), which is frequently used as a positive control for inducing pica behavior. All groups were provided free access to kaolin in their cages, and kaolin consumption was measured daily.

Methods

Animal Studies

Diet-induced obese scid mice: C57B1/6 scid (B6.scid, The Jackson Laboratory, Bar Harbor, ME, USA, stock no. 001913) male mice were fed with a high fat diet (Research diet, Cat #D12492, 60% fat kcal) for 12 weeks starting at 6 weeks of age. The mice were maintained at 22-23° C. with abundant bedding material, maintaining the animals in an environmental temperature close to their thermoneutrality, starting 2 weeks prior to the dosing period and for the full dosing period, with a 12 h/12 h light/dark cycle. Under these conditions, existing brown adipose atrophies and mice with a C57B1/6 background develop obesity, insulin resistance, and impaired glucose tolerance, with low to moderate hyperglycemia (diabetes). Only animals weighing≥40 g were used in the study to provide an adequate window to observe weight loss of 10% or more. Stratified randomization was employed to ensure that animals of similar weights were distributed across the study groups at the start of the study.

Mice were dosed every 3 days by intraperitoneal (IP) injection (100 μl per mouse) with either vehicle alone (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or EGS373 (3 mg/kg) dissolved in the vehicle for 28 days. As above, a third group received a single dose of cisplatin.

Kaolin intake, food intake, and body weight were measured daily. Food intake was measured by manually weighing the food in the hopper and any pieces left at the bottom of the cage on a daily basis.

Two days before the start and at the end of the dosing period, animals were fasted for 6 hours, and blood was collected. Plasma was isolated and saved at −20° C. until use for measurement of glucose, insulin and leptin levels (University of Cincinnati Mouse Metabolic Phenotyping Center). Body fat content was measured at the end of the dosing period by EchoMRT (University of Cincinnati Mouse Metabolic Phenotyping Center).

Statistical Analysis

Data from in vivo mouse studies are presented as mean±SEM. Significances were evaluated using unpaired Student's t-test, 2-way ANOVA or 1-way ANOVA with Bonferroni's or Dunnett's multiple comparison test using GraphPad Prism version 7 or 8 (GraphPad Software, San

Diego, CA). Significances were set at p<0.05, with *: p<0.05 vs. Vehicle, **: p<0.01 vs. Vehicle, ***: p<0.001 vs. Vehicle.

Results

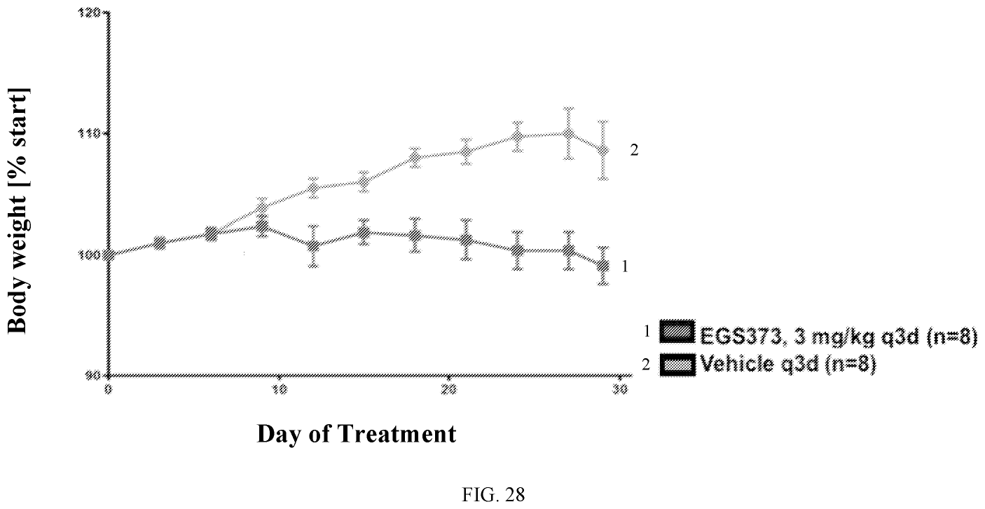

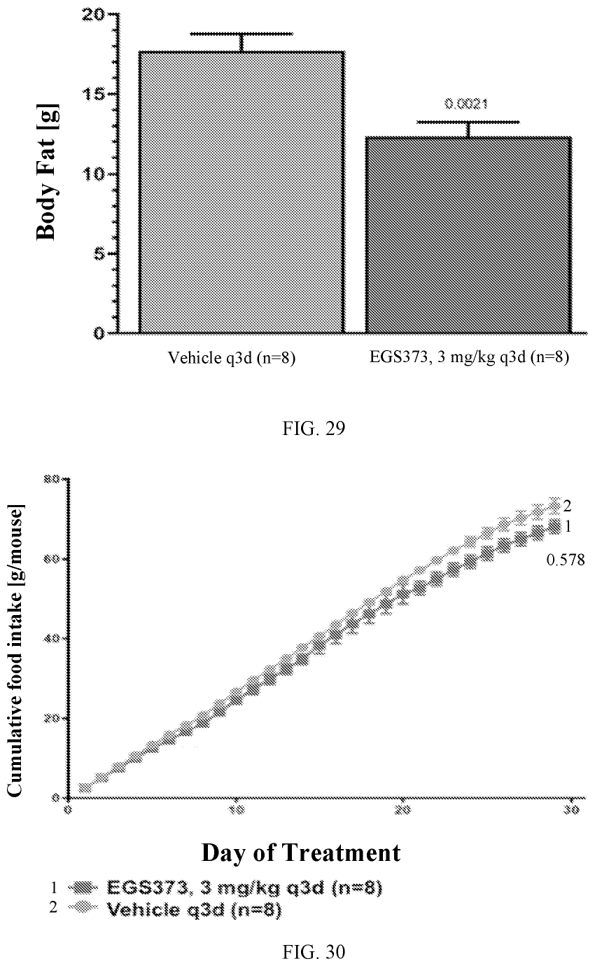

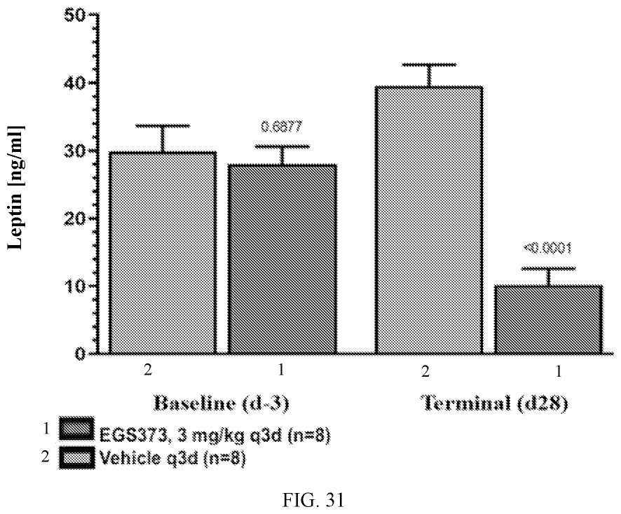

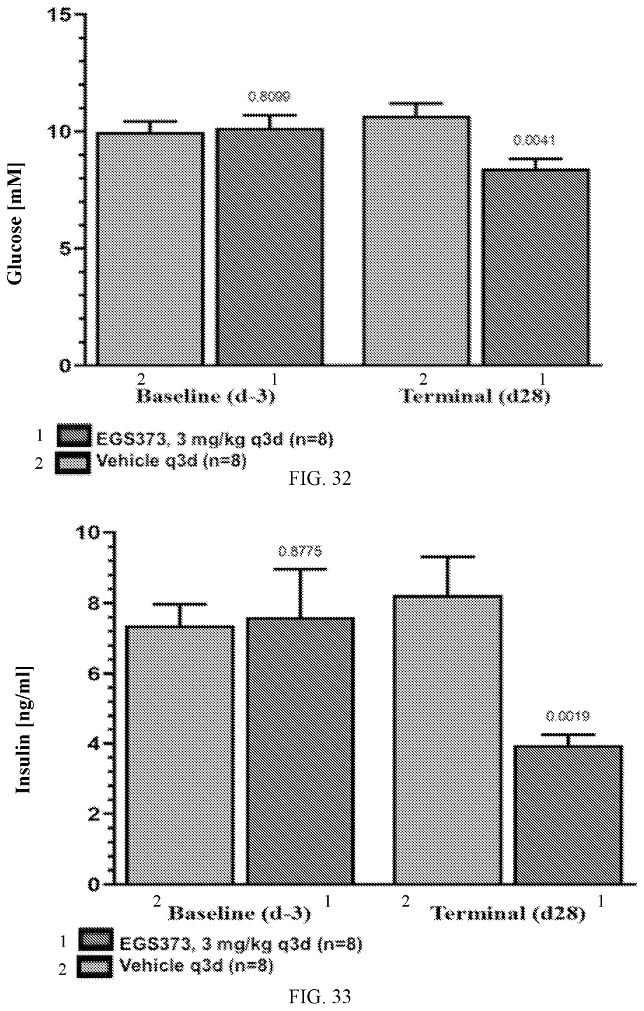

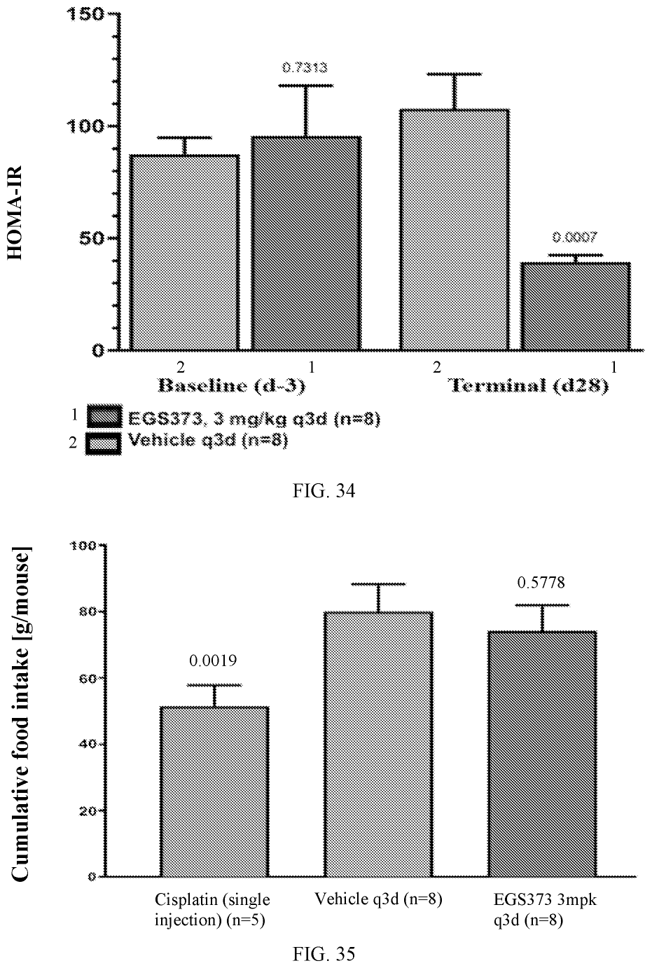

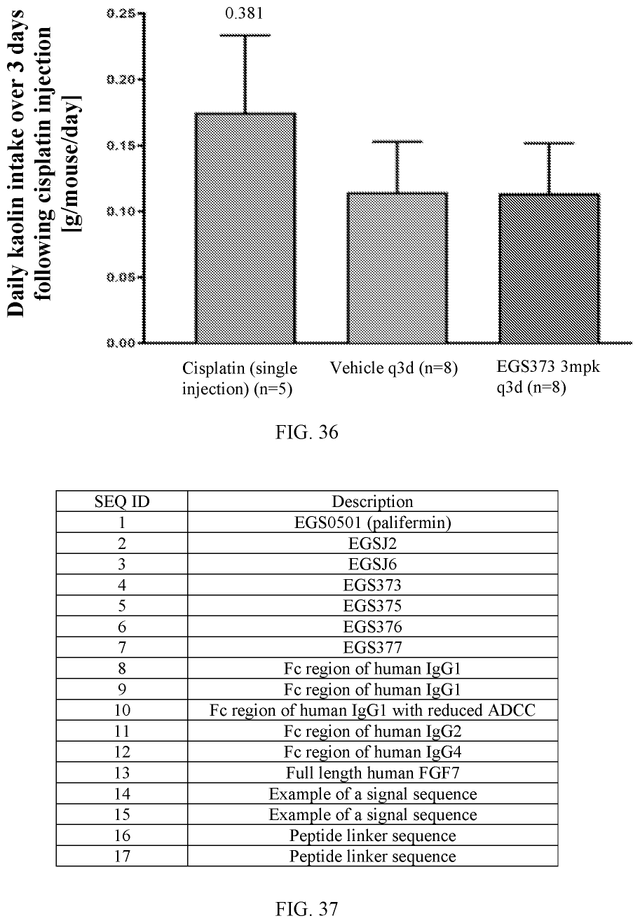

EGS373 induced significant body weight loss after 28 days of dosing (FIG. 28) due to losses of body fat (FIG. 29) with no significant effect on food intake (FIG. 30). The decrease in levels of plasma leptin (FIG. 31) induced by EGS373 confirmed the impact of the agent on body fat. The effects were accompanied by improvement in parameters of metabolic health: plasma levels of glucose (FIG. 32), insulin (FIG. 33), as well as index of insulin resistance, HOMA-IR (FIG. 34). While cisplatin reduced food intake (FIG. 35) and (non-significantly) induced kaolin consumption (FIG. 36), no corresponding pica behavior was induced by EGS373.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group baying 1-5 cells refers to groups having 1, 2, 3, 4, or 5 cells, and so forth.

All publications, patents and patent applications referenced in this specification are incorporated herein by reference in their entirety for all purposes to the same extent as if each individual publication, patent or patent application were specifically indicated to be so incorporated by reference. In case of conflict, the present specification, including definitions, will control.

SEQ ID NO: 1:
SYDYMEGGDIRVRRLFCRTQWYLRIDKRGKVKGTQEMKNNYNIMEIRTVAVGIVAIKGVES
EFYLAMNKEGKLYAKKECNEDCNFKELILENHYNTYASAKWTHNGGEMFVALNQKGIPVRG
KKTKKEQKTAHFLPMAIT
SEQ ID NO: 2:
CNDMTPEQMATNVNCSSPERHTRSYDYMEGGDIRVRRLFCRTQWYLRIDKRGKVKGTQEMK
NNYNIMEIRTVAVGIVAIKGVESEFYLAMNKEGKLYAKKECNEDCNFKELILENHYNTYAS
AKWTHNGGEMFVALNQKGIPVRGKKTKKEQKTAHFLPMAITAEPKSSDKTHTCPPCPAPEL
LGGPSVFLFPPKPKDTLMISRTPEVTCVVVDVSHEDPEVKFNWYVDGVEVHNAKTKPREEQ
YASTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 3:
APLEPKSSDKTHTCPPCPAPELLGGPSVFLFPPKPKDTLMISRTPEVTCVVVDVSHEDPEV
KFNWYVDGVEVHNAKTKPREEQYASTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGGGGSGGGGSC
NDMTPEQMATNVNCSSPERHTRSYDYMEGGDIRVRRLFCRTQWYLRIDKRGKVKGTQEMKN
NYNIMEIRTVAVGIVAIKGVESEFYLAMNKEGKLYAKKECNEDCNFKELILENHYNTYASA
KWTHNGGEMFVALNQKGIPVRGKKTKKEQKTAHFLPMAIT
SEQ ID NO: 4:
CNDMTPEQMATNVNCSSPERHTRSYDYMEGGDIRVRRLFCRTQWYLRIDKRGKVKGTQEMK
NNYNIMEIRTVAVGIVAIKGVESEFYLAMNKEGKLYAKKECNEDCNFKELILENHYNTYAS
AKWTHNGGEMFVALNQKGIPVRGKKTKKEQKTAHFLPMAITERKSSVECPPCPAPPVAGPS
VFLFPPKPKDTLMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYTLPPSREEMTKN
QVSLTCLVKGFYPSDISVEWESNGQPENNYKTTPPMLDSDGSFFLYSKLTVDKSRWQQGNV
FSCSVMHEALHNHYTQKSLSLSPGK
SEQ ID NO: 5:
APLERKSSVECPPCPAPPVAGPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTISK
TKGQPREPQVYTLPPSREEMTKNQVSLTCLVKGFYPSDISVEWESNGQPENNYKTTPPMLD
SDGSFFLYSKLTVDKSRWQQGNVFSCSVMHEALHNHYTQKSLSLSPGKGGGGSGGGGSCND
MTPEQMATNVNCSSPERHTRSYDYMEGGDIRVRRLFCRTQWYLRIDKRGKVKGTQEMKNNY
NIMEIRTVAVGIVAIKGVESEFYLAMNKEGKLYAKKECNEDCNFKELILENHYNTYASAKW
THNGGEMFVALNQKGIPVRGKKTKKEQKTAHFLPMAIT
SEQ ID NO: 6:
CNDMTPEQMATNVNCSSPERHTRSYDYMEGGDIRVRRLFCRTQWYLRIDKRGKVKGTQEMK
NNYNIMEIRTVAVGIVAIKGVESEFYLAMNKEGKLYAKKECNEDCNFKELILENHYNTYAS
AKWTHNGGEMFVALNQKGIPVRGKKTKKEQKTAHFLPMAITESKYGPPCPPCPAPEFLGGP
SVFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEGN
VFSCSVMHEALHNHYTQKSLSLSLG
SEQ ID NO: 7:
CNDMTPEQMATNVNCSSPERHTRSYDYMEGGDIRVRRLFCRTQWYLRIDKRGKVKGTQEMK
NNYNIMEIRTVAVGIVAIKGVESEFYLAMNKEGKLYAKKECNEDCNFKELILENHYNTYAS
AKWTHNGGEMFVALNQKGIPVRGKKTKKEQKTAHFLPMAITGGGGSESKYGPPCPPCPAPE
FLGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKSR
WQEGNVFSCSVMHEALHNHYTQKSLSLSLG
SEQ ID NO: 8:
DKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPG
SEQ ID NO: 9:
DKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQ
PREPOVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 10:
DKTHTCPPCPAPELLGGPSVFLFPPKPKDTLMISRTPEVTCVVVDVSHEDPEVKFNWYVDG
VEVHNAKTKPREEQYASTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 11:
VECPPCPAPPVAGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKTISKTKGQPREP
QVYTLPPSREEMTKNQVSLTCLVKGFYPSDISVEWESNGQPENNYKTTPPMLDSDGSFFLY
SKLTVDKSRWQQGNVFSCSVMHEALHNHYTQKSLSLSPGK
SEQ ID NO: 12:
ESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
SEQ ID NO: 13:
MHKWILTWILPTLLYRSCFHIICLVGTISLACNDMTPEQMATNVNCSSPERHTRSYDYMEG
GDIRVRRLFCRTQWYLRIDKRGKVKGTQEMKNNYNIMEIRTVAVGIVAIKGVESEFYLAMN
KEGKLYAKKECNEDCNFKELILENHYNTYASAKWTHNGGEMFVALNQKGIPVRGKKTKKEQ
KTAHFLPMAIT
SEQ ID NO: 14:
MEWSWVFLFFLSVTTGVHS
SEQ ID NO: 15:
ATGGAATGGAGCTGGGTCTTTCTCTTCTTCCTGTCAGTAACGACTGGTGTCCACTCC
SEQ ID NO: 16:
GGGGAGGGG
SEQ ID NO: 17:
GGGGSGGGG

### REFERENCES

Dumont J A, Low S C, Peters R T, Bitonti A J. 2006. Monomeric Fc fusions: impact on pharmacokinetic and biological activity of protein therapeutics. BioDrugs, 20, 151-60.

Timothy T. Kuo, Kristi Baker, Masaru Yoshida, Shuo-Wang Qiao, Victoria G. Aveson, Wayne I. Lencer, and Richard S. Blumberg. Neonatal Fc Receptor: From Immunity to Therapeutics. J Clin Immunol. 2010 November; 30(6): 777-789.

Boss O, Samec S, Kühne F, Bijlenga P, Assimacopoulos-Jeannet F, Seydoux J, Giacobino J P, and Muzzin P. Uncoupling protein-3 expression in rodent skeletal muscle is modulated by food intake but not by changes in environmental temperature. J Biol Chem 273: 5-8, 1998.

Lee P, Smith S, Linderman J, Courville A B, Brychta R J, Dieckmann W, Werner C D, Chen K Y, and Celi F S. Temperature-Acclimated Brown Adipose Tissue Modulates Insulin Sensitivity in Humans. Diabetes 63: 3686-3698, 2014.

Luu Y K, Lublinsky S, Ozcivici E, et al. In Vivo Quantification of Subcutaneous and Visceral Adiposity by Micro Computed Tomography in a Small Animal Model. Medical engineering and physics. 2009; 31(1):34-41. doi:10.1016/j.medengphy.2008.03.006.

Kolumam G, Chen M Z, Tong R, Zavala-Solorio J, Kates L, van Bruggen N, Ross J, Wyatt S K, Gandham V D, Carano R A, Dunshee D R, Wu A L, Haley B, Anderson K, Warming S, Rairdan X Y, Lewin-Koh N, Zhang Y, Gutierrez J, Baruch A, Gelzleichter T R, Stevens D, Rajan S, Bainbridge T W, Vernes J M, Meng Y G, Ziai J, Soriano R H, Brauer M J, Chen Y, Stawicki S, Kim H S, Comps-Agrar L, Luis E, Spiess C, Wu Y, Ernst J A, McGuinness O P, Peterson A S, and Sonoda J. Sustained Brown Fat Stimulation and Insulin Sensitization by a Humanized Bispecific Antibody Agonist for Fibroblast Growth Factor Receptor 1/betaKlotho Complex. EBioMedicine 2: 730-743, 2015.

J S Rubin, H Osada, P W Finch, W G Taylor, S Rudikoff, S A Aaronson. Purification and characterization of a newly identified growth factor specific for epithelial cells. Proc Natl Acad Sci USA, 86 (1989), pp. 802-806.

1-20. (canceled)

21. A method of identifying compound(s) which are Fc fusion proteins of human FGF-7, with improved half-life relative to EGS0501 for in vivo use in a human or animal.

22. A composition comprising SEQ ID NO: 2, SEQ ID NO: 3, SEQ ID NO: 4, SEQ ID NO: 5, SEQ ID NO: 6, or SEQ ID NO: 7, either alone or in combination, and a pharmaceutically acceptable carrier.

23. The pharmaceutical composition of claim 22, comprising SEQ ID NO: 2 and a pharmaceutically acceptable carrier.

24. The pharmaceutical composition of claim 22, comprising SEQ ID NO: 3 and a pharmaceutically acceptable carrier.

25. The pharmaceutical composition of claim 22, comprising SEQ ID NO: 4 and a pharmaceutically acceptable carrier.

26. The pharmaceutical composition of claim 22, comprising SEQ ID NO: 5 and a pharmaceutically acceptable carrier.

27. The pharmaceutical composition of claim 22, comprising SEQ ID NO: 6 and a pharmaceutically acceptable carrier.

28. The pharmaceutical composition of claim 22, comprising SEQ ID NO: 7 and a pharmaceutically acceptable carrier.

29. The pharmaceutical composition of claim 22, wherein said active ingredient is provided in therapeutically effective amounts that, when administered to a patient, are sufficient to treat or reduce obesity.

30. The pharmaceutical composition of claim 22, wherein said active ingredient is provided in therapeutically effective amounts that, when administered to a patient, are sufficient to treat or reduce type II diabetes.

31. The pharmaceutical composition of claim 22, wherein the therapeutically effective amount of said active ingredient in humans ranges from: a) about 0.1 mg/kg to about 1.0 mg/kg; or b) about 0.2 mg/kg to about 2 mg/kg.

32. The pharmaceutical composition of claim 22, wherein said active ingredient is provided in therapeutically effective amounts capable of inducing the expression of UCP1, FABP4 (aP2), PPARγ2, mtTFA, PGC-lα, and/or COX IV in BAT progenitor cells in human skeletal muscle, in vitro, in vivo, or both.

33. The pharmaceutical composition of claim 22, wherein said composition has one or more biological activities selected from the group consisting of:
(a) an increase in thermogenesis in brown adipose tissue and/or skeletal muscle tissue;
(b) an increase in insulin sensitivity of skeletal muscle, white adipose tissue, or liver;
(c) an increase in glucose tolerance;
(d) an increase in basal respiration, maximal respiration rate, or uncoupled respiration;
(e) an increase in metabolic rate;
(f) a decrease in hepatosteatosis;
(g) a decrease in body weight;
(h) a decrease in body fat mass;
(i) a decrease in plasma leptin levels;
(j) a decrease in glycemia;
(k) a decrease in plasma insulin levels;
(l) a decrease in insulin resistance;
or a combination thereof.

34. A method of promoting brown adipogenesis in a subject in need thereof, the method comprising administering to the subject the pharmaceutical composition of claim 22.

35. The method of claim 34, further comprising modulating a metabolic response in the subject and/or preventing or treating a metabolic disorder in the subject.

36. The method of claim 35, wherein the metabolic disorder is one or more of obesity, overweight, type II diabetes, insulin resistance, hyperinsulinemia, hyperglycemia, pre-diabetes, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.

37. The method according to claim 34, wherein the pharmaceutical composition comprises a therapeutically effective amount of said active ingredient in humans that ranges from: a) about 0.1 mg/kg to about 1.0 mg/kg; or b) about 0.2 mg/kg to about 2 mg/kg.


SEQUENCE LISTING (17 sequences)

SEQ ID NO: 1 - protein, 140 aa, Artificial Sequence
  truncated FGF-7
SYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 2 - protein, 396 aa, Artificial Sequence
  FGF-7 fusion protein
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 406 aa, Artificial Sequence
  FGF-7 fusion protein
APLEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 4 - protein, 391 aa, Artificial Sequence
  FGF-7 fusion protein
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 5 - protein, 404 aa, Artificial Sequence
  FGF-7 fusion protein
APLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 6 - protein, 391 aa, Artificial Sequence
  FGF-7 fusion protein
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 396 aa, Artificial Sequence
  FGF-7 fusion protein
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 226 aa, Artificial Sequence
  Fc region of human IgG1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 227 aa, Artificial Sequence
  Fc region of human IgG1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, Artificial Sequence
  Fc region of human IgG1 with reduced ADCC
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 223 aa, Artificial Sequence
  Fc region of human IgG2
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 12 - protein, 228 aa, Artificial Sequence
  Fc region of human IgG4
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 14 - protein, 19 aa, Artificial sequence
  signal
MEWSWVFLFFLSVTTGVHS

SEQ ID NO: 15 - DNA, 57 bp, Artificial sequence
  signal
atggaatggagctgggtctttctcttcttcctgtcagtaacgactggtgtccactcc

SEQ ID NO: 16 - protein, 9 aa, Artificial Sequence
  peptide linker
GGGGAGGGG

SEQ ID NO: 17 - protein, 9 aa, Artificial Sequence
  peptide linker
GGGGSGGGG